(19) United States
(12) Patent Application Publication
(10) Pub. No.: US 2015/0096561 A1
(43) Pub. Date: Apr. 9, 2015

(54) DEVICES AND METHODS FOR AIRWAY MANAGEMENT

(71) Applicant: The Board of Trustees of The Leland Standard Junior University, Stanford, CA (US)

(72) Inventors: Mary Katherine GARRETT, Redwood City, CA (US); Dan E. AZAGURY, Geneve (CH); Gary B. HULME, San Jose, CA (US); Ronan L. JENKINSON, Pittsburg, CA (US)

(73) Assignee: The Board of Trustees of The Leland Standard Junior University, Stanford, CA (US)

(21) Appl. No.: 14/507,377

(22) Filed: Oct. 6, 2014

Related U.S. Application Data
(60) Provisional application No. 61/887,560, filed on Oct. 7, 2013.

(51) Int. Cl. A61M 16/04

(52) U.S. Cl. CPC A61M 16/0438; A61M 16/0488
USPC 128/202.27; 128/207.15

(57) ABSTRACT

Devices and methods for airway management are described herein where a barrier may be delivered and deployed along an endotracheal tube to prevent aspiration or passage of fluids and debris into the trachea in patients who are intubated. Generally, the barrier apparatus may comprise an expandable barrier assembly configured to be positioned over or around an endotracheal tube, a pushing assembly having a handle and a first shaft extending therefrom, and a holding member securable to the endotracheal tube and slidingly engagable with the pushing assembly.

### CROSS-REFERENCE TO RELATED APPLICATIONS

This application claims the benefit of priority to U.S. Provisional Application No. 61/887,560 filed Oct. 7, 2013 which is incorporated herein by reference in its entirety.

### FIELD OF THE INVENTION

The present invention relates generally to devices and methods for limiting tracheal aspiration, which may help prevent conditions such as ventilator associated pneumonia (“VAP”) in patients who are intubated.

### BACKGROUND OF THE INVENTION

Patients who are intubated with an endotracheal (ET) tube are typically intubated to facilitate mechanical ventilation but are also associated with an increase in morbidity. Because ET tubes generally utilize an inflatable balloon between the tube and the walls of the trachea to prevent aspiration or passage of fluids and debris into the trachea, small pools of pathogen-containing secretions may pool in the sub-glottic space. Small channels sometimes develop between the balloon walls and the walls of the trachea through which debris and subglottic secretions pass into the lower respiratory tract.

Prior devices have attempted to clear or prevent the aspiration of the debris and secretions such as endotracheal tubes which drain the fluid via suction; however, such devices may require intermittent suctioning and further require specialized ET tubes. Other previous devices have utilized balloon cuff designs which have varying configurations yet such devices require the purchase and use of these specific ET tubes. These devices utilizing ET tubes require their use at the time of intubation and indiscriminately across all patients which make them prohibitively expensive for many medical facilities.

Additional prior devices have utilized the infusion or introduction of obstructing materials such as gels, foams, polymers, etc. in the sub-glottic space in proximity to the inflatable ET balloon or in place of the balloon itself. However, such materials may present difficulty in removal from the patient's airway and may also present the possibility of inadvertent aspiration itself.

Accordingly, there exists a need for devices and methods which allow for rapid deployment into a patient and which may also be used in conjunction with conventional ET tubes which are already in wide use.

### BRIEF SUMMARY OF THE INVENTION

In patients intubated with an endotracheal (“ET”) tube, an expandable barrier member may be delivered along or over the ET tube and deployed in proximity to the ET balloon to form a barrier between an outer surface of the ET tube and the surrounding tissue walls of the patient's airway. The barrier member may be positioned anywhere along the ET tube, such as in the larynx or trachea, and may form a barrier between the ET tube and the surrounding anatomy (e.g., the vestibule of the larynx, the vocal cords, or the like). Such a barrier may prevent microaspiration or aspiration of material and/or bacteria through the vocal cords and into the trachea, which may in turn result in VAP or aspiration pneumonia.

When the devices and methods described herein are used to create a barrier along the ET tube, the devices may be used with any number of conventional or specifically-configured ET tubes. The barrier member may be positioned simultaneously with the ET tube or positioned after the patient has already been intubated with the ET tube. In some of these instances, a barrier member as described herein may be delivered after a patient has been intubated for a given period of time. In other instances, the barrier member may be delivered when a balloon of the ET tube has failed or otherwise deflated such that the barrier member may provide a seal in place of the failed ET balloon.

Generally, one variation of the barrier apparatus for use with an endotracheal tube may comprise an expandable barrier assembly configured to be positioned over or around an endotracheal tube, a pushing assembly having a handle and a first shaft extending therefrom, the first shaft having a length which defines a curvature configured to conform to an airway of a patient and a distal end configured to engage at least a portion of the barrier assembly, and a holding member securable to the endotracheal tube and slidingly engagable with the pushing assembly.

In one method of use, the delivering and deploying the barrier apparatus may generally comprise securing a holding member along a portion of an endotracheal tube extending from a mouth of a patient, urging a first shaft and optionally a second shaft distally relative to the holding member such that the first shaft and optional second shaft follow the endotracheal tube until a barrier assembly is positioned via the first shaft within about 0-5 cm in proximity to or at least partially past the vocal cords of the patient, releasing the first shaft from the second shaft, and, further advancing the first shaft relative to the holding member such that the barrier assembly is positioned distal to the vocal cords of the patient.

### BRIEF DESCRIPTION OF THE DRAWINGS

FIG. 1 shows an example of an ET tube which may be positioned in the airway of a patient.

FIG. 2 depicts a side view of an illustrative variation of the systems described here.

FIG. 3A shows a perspective view of a variation of a balloon cuff assembly suitable for use with the systems described here.

FIGS. 3B and 3C show top perspective and bottom perspective views of a variation of a balloon cuff of the balloon cuff assembly of FIG. 3A.

FIGS. 4A and 4D show a perspective view of an illustrative variation of a pushing assembly suitable for use with the delivery devices described here.

FIGS. 4B and 4C depict perspective views of portions of the pushing assembly of FIGS. 4A and 4D.

FIGS. 5A-5C depict perspective, rear, and side views, respectively, of a variation of a holding member suitable for use with the systems described here.

FIGS. 6A-6C depict side views of a variation of a holding member as described here releasably connected to a variation of a pushing assembly as described here.

FIGS. 7A-7C depict front, side, and top views, respective, of a variation of a loading assembly suitable for use with the systems described here.

FIGS. 8A and 8B depict an illustrative method of using a variation of the loading assemblies described here to load a barrier member onto an ET tube.

FIG. 9 depicts a variation of a loading assembly as described here connected to a variation of a holding member as described here.

FIGS. 10A-10G depict an illustrative variation of a method of positioning a balloon via a delivery device along an ET tube positioned within a patient.

FIGS. 11A-11C depict perspective views of alternative embodiments of the distal end of a pushing assembly suitable for use with the delivery devices described here.

FIGS. 12A and 12C depict side views of a variation of a holding member as described here connected to a variation of a pushing assembly as described here.

FIG. 12B depicts a front view of a variation of a holding member as described here connected to a variation of a pushing assembly as described here.

FIGS. 13A and 13B depict perspective and front views of another variation of a balloon cuff assembly suitable for use with the systems described here.

FIGS. 14A and 14B depict side views of another variation of a pushing assembly suitable for use with the delivery devices described here.

FIGS. 15A to 51D depict respective side, end, top, perspective views of yet another variation of a balloon cuff assembly suitable for use with the delivery devices described herein.

FIGS. 16A and 16B depict top and perspective views of the balloon cuff assembly of FIG. 15A having a balloon inflated upon the assembly.

FIG. 17 depicts a perspective view of the balloon cuff assembly of FIG. 15A engaged with a second shaft configured as a relatively flattened ribbon member.

FIGS. 18A and 18B depict side views of a pushing assembly having a balloon cuff assembly engaged and advanced distally by the first shaft and second shaft.

### DETAILED DESCRIPTION OF THE INVENTION

Described here are devices and methods for preventing or reducing the risk for aspiration in intubated patients. One embodiment comprises forming a barrier within or along a patient's airway. This barrier may act to prevent liquid, solid, or particulate matter from traveling past the barrier. For example, a patient may be intubated with an endotracheal (“ET”) tube, and the devices described here may deploy a barrier member along the ET tube to form a barrier between the ET tube and the patient's airway. The barrier member may be positioned anywhere along the ET tube, such as in the larynx or trachea, and may form a barrier between the ET tube and the surrounding anatomy (e.g., the vestibule of the larynx, the vocal cords, or the like). Such a barrier may prevent microaspiration or aspiration of material and/or bacteria through the vocal cords and into the trachea, which may in turn result in VAP or aspiration pneumonia.

When the devices and methods described here are used to create a barrier along an ET tube, the devices may be used with any number of conventional or specifically-configured ET tubes. The barrier member may be positioned simultaneously with the ET tube, or may be positioned after the patient has already been intubated with the ET tube. In some of these instances, a barrier member as described here may be delivered when a patient has been intubated for a given period of time (e.g., if the patient requires ventilator assistance for more than a day), it may be desirable to deploy a barrier member to supplement the barrier provided by the balloon of the ET tube. In other instances, a barrier member as described here may be delivered when a balloon of the ET tube has failed or otherwise deflated, such that the barrier member may provide a seal in place of the failed ET balloon.

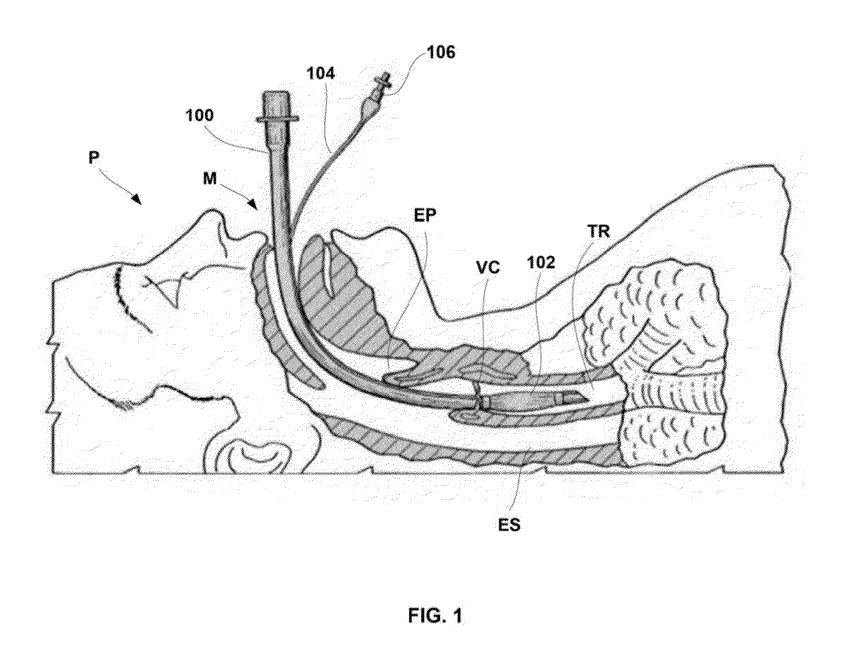

For the purposes of illustration, FIG. 1 illustrates the anatomy of the upper airway of a patient intubated with an ET tube (100). To position the ET tube (100) as shown in FIG. 1, the ET tube (100) may be inserted through the mouth (M) of a patient (P), and may be advanced past the epiglottis (EP) into the trachea (TR). Also shown there is the esophagus (ES). The ET tube (100) may comprise an ET balloon (102). Generally, the ET balloon (102) may be positioned in the trachea (TR) past the vocal cords (VC). The ET tube (100) may further comprise an ET inflation tube (104) coupled to the ET balloon (102) and fluidly coupling the ET balloon (102) to an ET inflation port (106). Gas or fluid may be inserted into the ET balloon (102) through the ET inflation tube (104) and the ET inflation port (106) to inflate the ET balloon (102) in the trachea (TR), and gas or fluid may be withdrawn from the ET balloon (102) through the ET inflation tube (104) and the ET inflation port (106). For example, a syringe or other fluid reservoir (not shown) may be connected to the ET inflation port (106) to inflate or deflate the ET balloon (102).

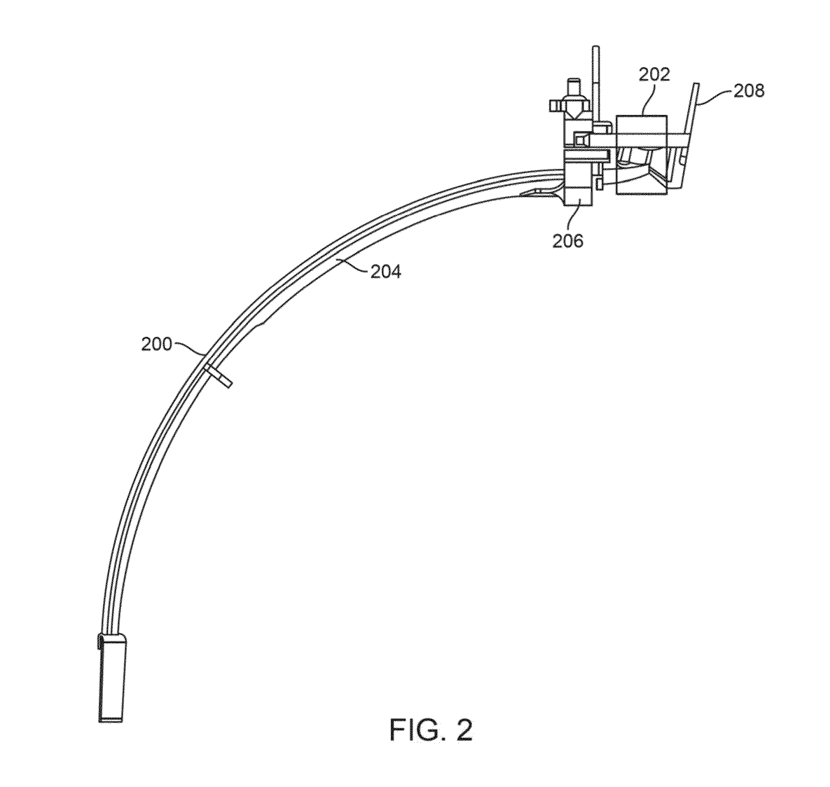

Generally, the systems described here may comprise a barrier assembly and a delivery device for deploying the barrier assembly. In some variations, the barrier assembly may comprise a balloon cuff assembly, although it should be appreciated that the barrier assembly may be any member capable of forming a barrier in the airway of a patient. FIG. 2 shows a side view of an illustrative variation of a system as described here. As shown there, the system may comprise a delivery device (200) and a barrier assembly (202). While the barrier assembly (202) is illustrated as one embodiment in FIG. 2, the barrier assembly (202) may take on any number of configurations which are suitable for performing the functions described herein. The barrier assembly (202) may include a barrier member, which may be inserted over or around an ET tube or other device (e.g., a laryngeal mask). In some variations, the barrier member may be inflatable or otherwise expandable, and may be selectively inflated or expanded in a patient's airway to form a barrier therein. In some of these variations, the barrier assembly may comprise a balloon cuff assembly, as will be described in more detail below. In other variations, the barrier assembly may comprise a foam member, a viscous gel or liquid, a sponge, or the like.

Returning to FIG. 2, the delivery device (200) may comprise a pushing assembly (204), a holding member (206), and a loading assembly (208). Generally, the pushing assembly (204) may be configured to engage the barrier member, and to push, pull, or otherwise advance the barrier member into the airway of a patient. The holding member (206) may be connected to the pushing assembly (204), and may be configured to temporarily connect the pushing assembly (204) to an ET tube (or other similar device). The loading assembly (208) may also releasably connect to the barrier assembly (202) and/or the holding member (206), and may facilitate loading the barrier assembly (202) and/or the holding member (206) onto an ET tube (or other similar device), such as described in more detail below. While the systems are generally described here as being used to advance a barrier member along an ET tube, it should be appreciated that the systems may be used to advance a barrier member along any suitable generally tubular device, such as a laryngeal mask, an endoscope, or the like.

While shown in FIG. 2 as having each of a pushing assembly (204), a holding member (206), and a loading assembly (208), it should be appreciated that the delivery devices described here need not include all of these components. For example, in some variations, the delivery device may include a pushing assembly, but may not include a holding member or a loading assembly. In other variations, a delivery device may include a pushing assembly and a loading assembly, but not a holding member. In other variations, the delivery device may include a pushing assembly and a holding member, but not a loading assembly.

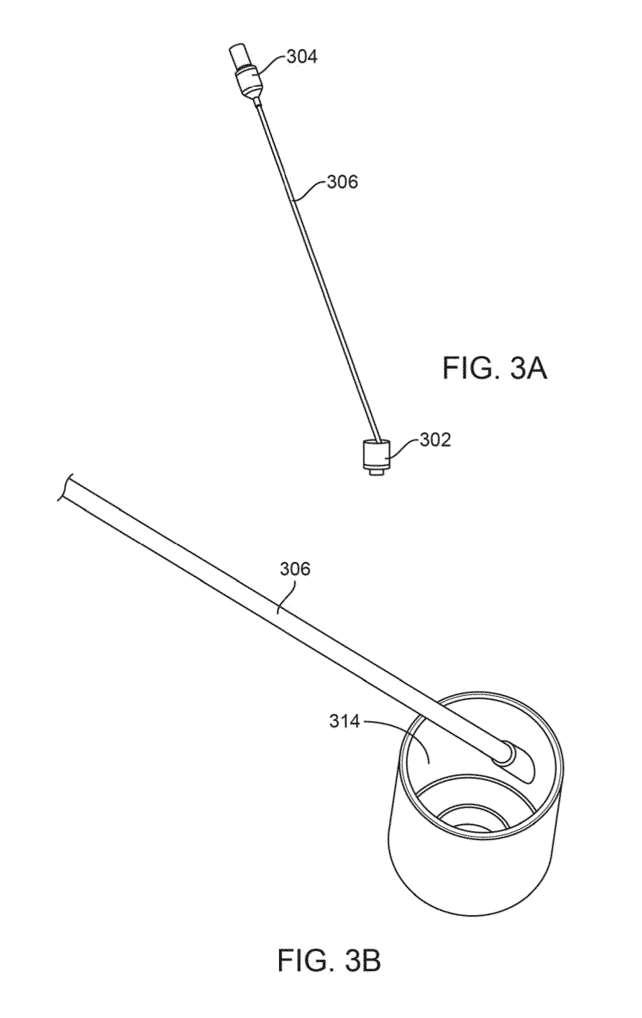

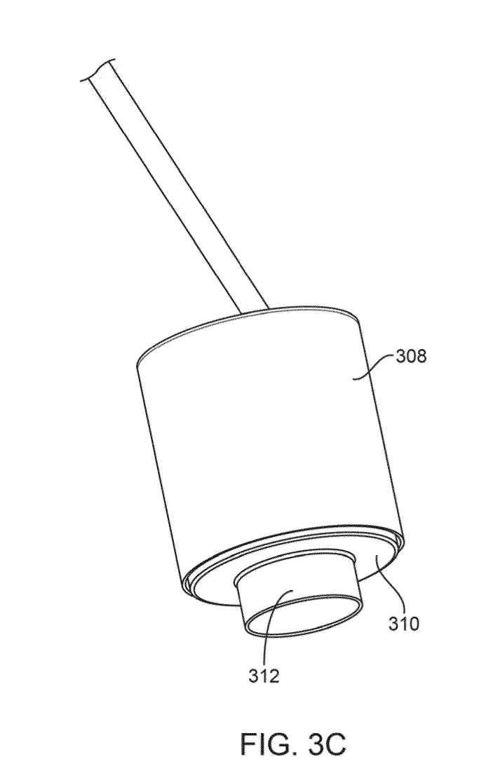

As mentioned above, in some variations the delivery devices described here may deliver any suitable barrier member to a patient. The barrier member may be any suitable member, such as, for example, one or more of the deployable members described in U.S. patent application Ser. No. 13/714,124, filed on Dec. 13, 2012 and titled “DEVICES AND METHODS FOR PREVENTING TRACHEAL ASPIRATION,” the content of which is hereby incorporated by reference in its entirety and for any purpose herein. In some variations, the barrier member may comprise a balloon cuff. For example, FIGS. 3A-3C depict a variation of a balloon cuff assembly (300) suitable for use with the systems described here. As shown there, the balloon cuff assembly (300) may comprise a balloon cuff (302), an inflation port (304), and an inflation tube (306) connecting the balloon cuff (302) and the inflation port (304). Gas or fluid may be introduced into or removed from the balloon cuff (302) via the inflation port (304) and the inflation tube (306) to inflate or deflate, respectively, the balloon cuff (302).

The balloon cuff (302) may have any suitable configuration of elements. For example, in the variation shown in FIGS. 3A-3C, the balloon cuff (302) may comprise a balloon (308), a support ring (310), and a sealing sleeve (312). These components may, in turn, define a lumen (314) extending through the balloon cuff (302). An ET tube may be inserted through the lumen (314) (such as described in more detail below) to load the balloon cuff (302). Generally, the balloon (308) may be fluidly connected to the inflation tube (306), which may allow the balloon to be inflated or deflated such as discussed above. The balloon may comprise any number of suitable biocompatible materials (e.g., silicone, urethane, combinations thereof and the like), and may be sized and shaped depending on the anatomy of the patient and the ET tube, laryngeal mask, or other device used with the balloon cuff assembly (300).

While shown in FIGS. 3A-3C as having a support ring (310), the balloon cuff (302) need not include a support ring (310). In variations where the balloon cuff (302) comprises a support ring (310), the support ring (310) may be configured to be attached to the balloon (308) and to provide structural support to the balloon (308). For example, the support ring (310) may be formed from a rigid material such as stainless steel sheeting, wire or ribbon, silicone, rubber, plastic, flexible fiber mesh, and may be engaged by a portion of a delivery device to advance the balloon cuff (302) along an ET tube. The support ring (310) may be formed integrally with the balloon (308), or may be formed separately from the balloon (308) and connected (e.g. via bonding, welding, or the like) to the balloon (308). While the support ring (310) is shown in FIGS. 3A-3C as fully extending around the lumen (314) of the balloon cuff (302), it should be appreciated that in some variations the support ring (310) may extend partially around the lumen.

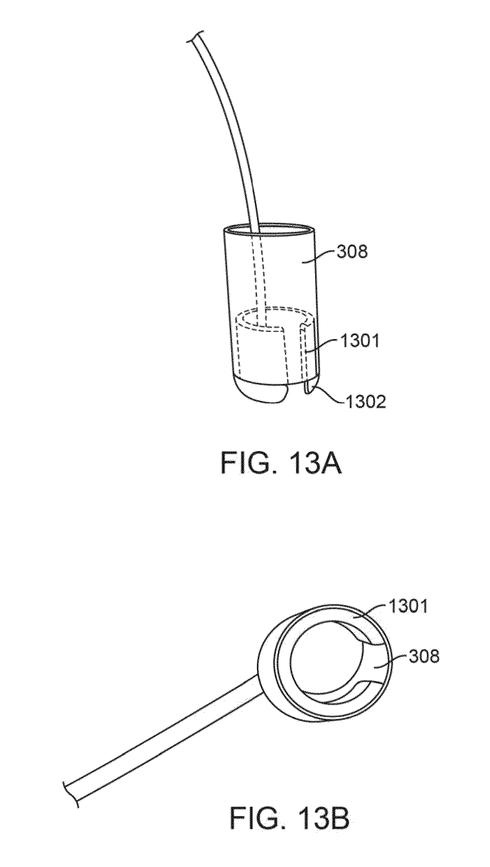

FIGS. 13A and 13B show perspective and front views, respectively, of another example of the balloon cuff (302) comprising a balloon (308), (shown in FIGS. 13A and 13B in a deflated low-profile configuration), and a support ring (1301). In some variations, such as shown in FIGS. 13A and 13B, the support ring (1301) may have a tapered distal tip (1302) which may allow for atraumatic advancement through the airway anatomy when the balloon is mounted on an ET tube. The proximal end of the support ring may also be tapered or otherwise shaped. Additionally, as shown in FIGS. 13A and 13B, the support ring (1301) may extend partially around the balloon (308), which may allow the support ring (1301) to flex and accommodate variations in ET tube diameter. In some variations, such as the embodiment shown in FIGS. 13A and 13B, the balloon (308) is directly mounted on the support ring (1301). In the variations, expansion of the support ring (1301) may cause the balloon (308) to flex along with the support ring (1301). In other variations, the balloon cuff (302) may comprise an elastic sleeve connecting the balloon to the support ring. In these variations, expansion of the support ring may also expand the elastic sleeve, but the balloon need not expand (although it should be appreciated that in some instances both the balloon and the elastic sleeve may expand). The balloon (308) could be larger than the ETT and non-compliant.

As further examples, the support ring (310) may be any of the inflation rings described in U.S. patent application Ser. No. 13/714,124, which was previously incorporated by reference in its entirety.

Additionally, while shown in FIGS. 3A-3C as having a sealing sleeve (312), the balloon cuff (302) need not include a sealing sleeve (312). In variations where the balloon cuff (302) comprises a sealing sleeve (312), the sealing sleeve may be attached to the balloon (308) and/or the support ring (310) (in variations where the balloon cuff (302) comprises a support ring (310)). The sealing sleeve (312) may be formed from an elastic or distensible material (e.g., urethane, or the like), such that the sealing sleeve (312) may seal against a member positioned in the lumen (314) of the balloon cuff (302). For example, in variations where the balloon cuff (302) may be loaded onto an ET tube, the sealing sleeve (312) may be have a diameter less than or equal to the ET tube. When the ET tube is positioned within the lumen (314) of the balloon cuff (302), the sealing sleeve (312) may conform to the outer surface of the ET tube and form a seal between the balloon cuff (302) and the ET tube.

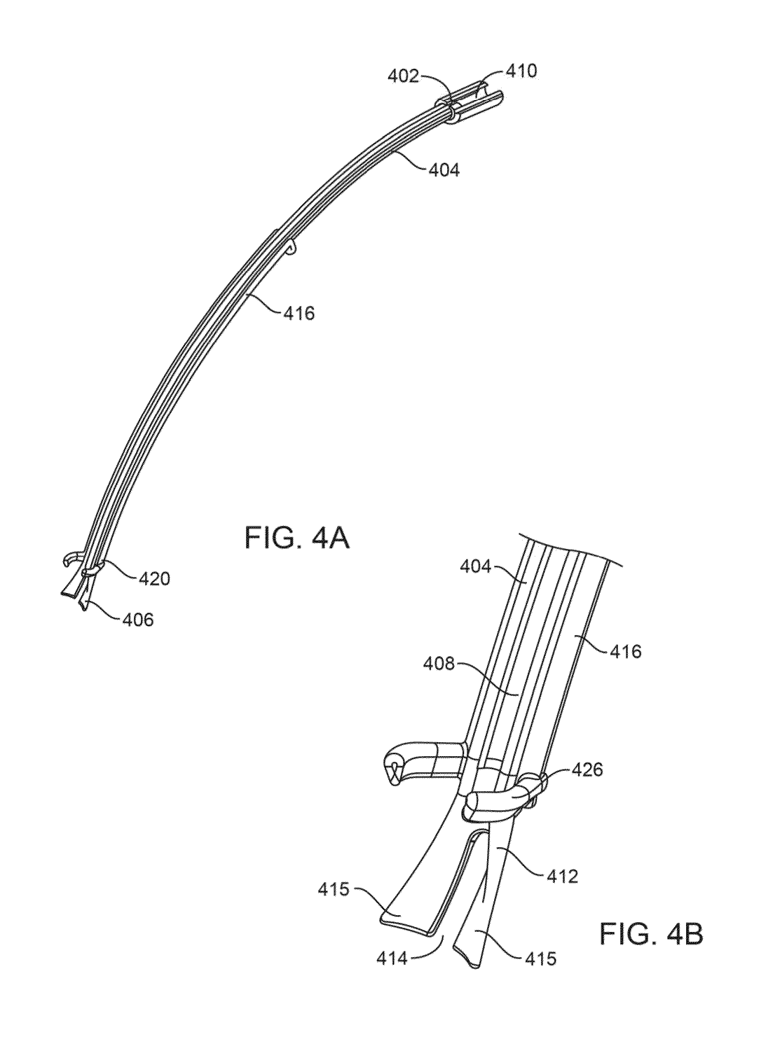

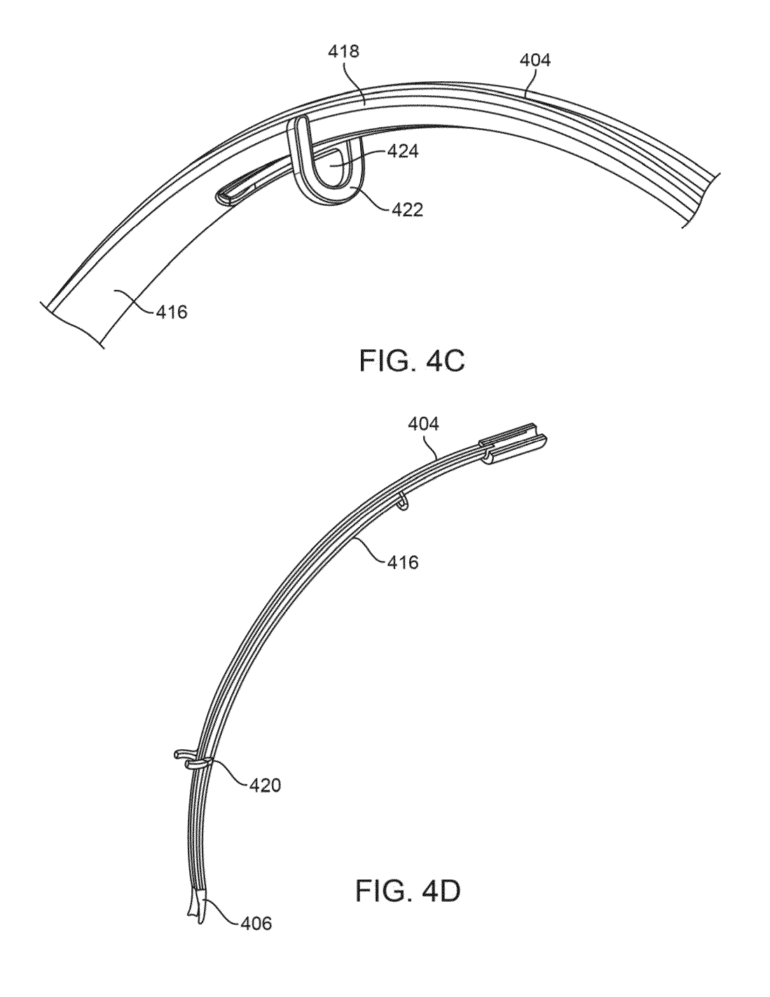

As mentioned above, the delivery devices described here may comprise a pushing assembly. Generally, the pushing assembly may comprise a handle and one or more shafts. FIGS. 4A-4D depict a variation of a pushing assembly (400) suitable for use with the systems described here, such as the delivery device (200) shown in FIG. 2 above. As shown there, the pushing assembly (400) may comprise a handle (402), and a first shaft (404) connected to and extending from the handle (402). A distal portion (406) of the first shaft (404) may be configured to engage a portion of a barrier member (e.g., a balloon cuff assembly), and may provide a pushing force to the barrier member to advance the barrier member along an airway of a patient.

The first shaft (404) is preferably curved during advancement of a barrier member, but need not be. In some variations, the first shaft (404) may have a permanent curvature (e.g., may be pre-formed with one or more curves). In other variations, the first shaft (404) may be flexible, such that the first shaft (404) may take on a specific curvature or otherwise conform to the patient's anatomy when a pushing force is applied to the handle (402). The first shaft (404) may have any suitable radius of curvature (e.g., 8 in, between 6 in and 30 in, or the like), and may have any suitable length (e.g., between 5 in and 20 in, or the like). A curved first shaft may present an atraumatic profile to the anatomy of the trachea during advancement of the first shaft (404).

In some variations, the first shaft (404) may comprise a channel (408) extending along at least a portion of the first shaft (404), but need not. In variations that do include the channel (408), the channel (408) may extend along the length of the first shaft (404) between the handle (402) and the distal portion (406), and may be configured to receive a portion of a barrier member assembly. For example, in variations where the barrier member assembly is a balloon cuff assembly comprising inflation tubing, the channel (408) may be configured to secure or otherwise hold the inflation tube relative to the first shaft (404). For example, a balloon cuff assembly may be positioned such that the distal portion (406) of the first shaft engages a balloon cuff of the assembly, the handle (402) engages an inflation port of the assembly (as discussed below), and the inflation tube may be positioned along the first shaft (404) such that it is received by the channel (408). In some variations, this may tension the inflation tube, which may naturally hold the inflation tube and the channel. In other variations, the channel (408) may be configured to form a friction fit with the inflation tube when the inflation tube is positioned along first shaft (404).

Generally, a user may manipulate the handle (402) to control movement of the first shaft (404) relative to a patient. For example, a user may apply a pushing force to the handle (402) to advance the distal portion (406) of the first shaft (404) relative to a patient, which may in turn advance a barrier member. In some variations, the handle (402) may comprise a recess (410) configured to receive an inflation port of a balloon cuff assembly, which may temporarily couple the inflation port to the handle (402). In some variations, the recess (410) may be sized and configured to form a friction fit with the inflation port when the inflation port is positioned in the recess (410). Additionally or alternatively, the recess (410) may comprise on or more latches or covers (not shown) to hold the inflation port in the recess (410). The handle (402) may also incorporate a button or release (not shown) which may be optionally depressed or actuated to eject the inflation port from the recess (410) and the handle (402). In some variations, the recess (410) may be configured to align the inflation port with one or more fluid reservoirs, such as syringe, which may be coupled to the inflation port to introduce or remove fluid or gas from a balloon cuff.

The distal portion (406) of the first shaft (404) may be configured to engage a portion of a barrier member (such as balloon cuff), such that a pushing force applied to handle (402) may cause the distal portion (406) to advance the barrier member. For example, FIG. 4B shows a larger view of a variation of a distal portion (406) of the first shaft (404). In the variation shown there, the distal portion (406) may comprise a forked member (412) with a slot (414) positioned between two tines (415). The tines (415) may be configured to contact and press against one or more portions of a barrier member. For example, when the pushing assembly (400) is used to advance a balloon cuff having a balloon and a support ring (and optionally a sealing sleeve, as discussed above), such as the balloon cuff (302) described above with respect to FIGS. 3A-3C, the tines (415) may be configured to engage and push against the support ring of the balloon cuff to advance the balloon cuff (e.g., along an ET tube or the like). In some variations, the tines (415) may be curved, but need not be. In some of these variations, the tines (415) may have a radius of curvature approximately equal to that of the support ring. Additionally, in variations where the balloon cuff is attached to an inflation tube (e.g., the inflation tube (306) of the balloon cuff assembly (300) described above with respect to FIGS. 3A-3C), the slot (414) may be positioned such that the inflation tube extends through the slot (414). In variations where the first shaft (404) comprises a channel (408) extending along the length of the first shaft (404), the slot (414) may align the inflation tube with the channel (408) to facilitate positioning of the inflation tube in the channel (408).

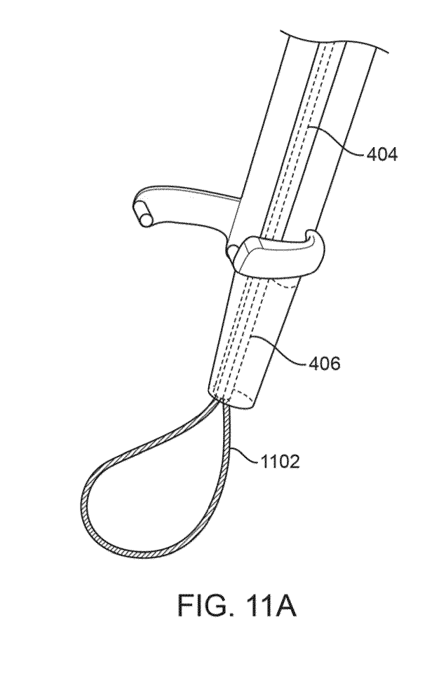

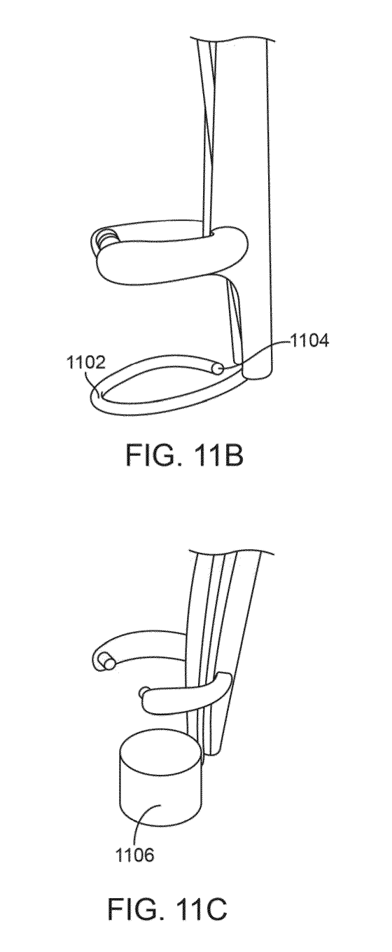

While shown in FIGS. 4A and 4B as having a forked member (412), the distal portion (406) of the first shaft (404) may include any elements or elements which may be configured to engage and advanced a portion of a barrier member. FIGS. 11A-11C show alternative embodiments of the distal portions of a first shaft (such as the first shaft (404) shown in FIGS. 4A-4D, with identical components labeled as in FIGS. 4A-4D) that may be configured to engage a portion of a barrier member (such as balloon cuff), such that a pushing force applied to handle (402) may cause the distal portion to advance the barrier member. FIG. 11A shows a loop (1102) that extends from the distal portion (406) of the first shaft (404). In some variations, the first shaft may be a hypotube (e.g., a stainless steel hypotube or the like). This loop may be made of materials including wire (flat or round), wire rope, or polymer fiber. Multiple wires or fibers may be utilized together. One or both ends of the loop material may extend proximally to the handle (402) or somewhere along the length of the first shaft (404) allowing the loop diameter to be increased or decreased by pulling on the proximal end(s) of the loop material. This would allow the user to accommodate a range of endotracheal tube or barrier member sizes. Additionally, this may allow the user to pull the loop against an ET tube and/or balloon cuff assembly. For example, tightening the loop around the ET tube or balloon cuff assembly would reduce the flexibility of the loop. The increased rigidity of the loop may allow the loop to push against or otherwise advance the balloon cuff relative to the ET tube. For example, the loop may be positioned inside the balloon cuff assembly and tightened around the ET tube, and the loop may be advanced to push against the balloon or support ring such that advancement of the loop advances the balloon cuff.

FIG. 11B shows another variation of a distal portion of a first shaft (404). As shown there, a wire or fiber may be configured to form a partial loop (1102). Specifically, one end of the loop (1102) may be open as shown in FIG. 11B, which may allow the free end of the loop (1104) to expand to accommodate a range of endotracheal tube or barrier member sizes. In these variations, the loop may be rigid enough such that it may push against a portion of a balloon cuff to advance the balloon cuff, but may be flexible enough to allow for expansion of the loop to accommodate a larger loop. FIG. 11C shows an embodiment where a partial or complete ring (1106) is used as the distal portion of the first shaft. In this instance thin walled metal, rubber, or plastic, of wall thickness 0.001″-0.060″ may be used to create this ring. The height of this ring may be 1 mm to 20 mm in length.

For any of these embodiments shown in FIGS. 4A-4D and 11A-11C, when used with a balloon assembly as shown in FIGS. 3A-3C, the distal portion of the first shaft may engage one or more portion of the balloon assembly including but not limited to the balloon (308), the sealing sleeve (312), the support ring (310) or the inflation tubing (306), such that when the pusher is advanced the distal portion of the first shaft pushes against the balloon cuff assembly to move it forward.

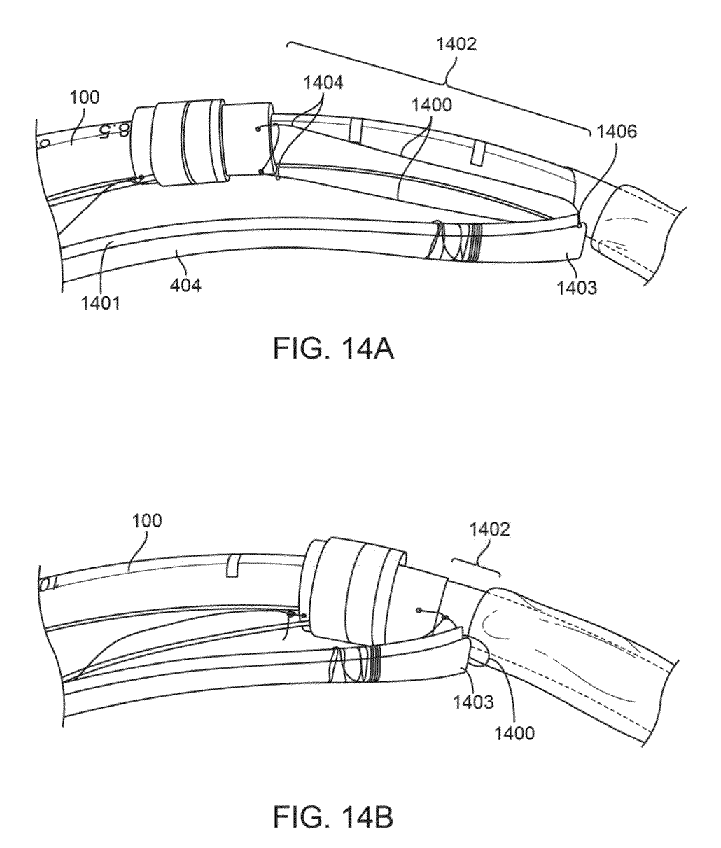

As an alternative to pushing, the first shaft (404) may be used to pull the barrier member distally down the ET tube (100) when positioned within the patient's body. FIGS. 14A and 14B depict side views of a first shaft (404) which utilizes one or more cables (1400). In this embodiment, the one or more cables (1400) may extend from the proximal end (not shown) of the first shaft (404) through cable lumens (1401), which may be optionally enclosed, down to the distal end (1403) of the first shaft (404). While the cables (1400) are shown in FIGS. 14A and 14B as exiting the distal end (1403) of the first shaft (404), in other variations one or more cables (1400) may exit the first shaft (404) along the length of the first shaft (404). The one or more cables (1400) may exit the cable lumens (1401) near or at the distal end (1406) of the cable lumen (1401) and may be connected to the barrier member at one or more attachment points (1404). As shown in FIGS. 14A and 14B, the barrier member may be positioned proximally of the distal end (1403) of the first shaft (404), which may allow one or more cables (1400) to pull the barrier member along the ET tube (100). The length of the one or more cables (1400) from the distal end (1406) of the cable lumen (1401) to the attachment point (1404) is illustrated as the exposed length of cable (1402). The one or more cables (1400) may be attached to any portion of the barrier member. In this embodiment, when the one or more cables (1400) are pulled in tension at the proximal end of the first shaft (not shown) external to the patient, the exposed length of cable (1402) will shorten, thereby advancing the barrier assembly towards the distal end (1403) of the first shaft (404). The distance traveled by the barrier assembly may be controlled by the length the user pulls the one or more cables (1400) or limited by the device.

Furthermore, the first shaft (404) may be advanced past the vocal cords of the patient, at which point the user may pull the one or more cables (1400) in tension, shortening the exposed length of cable (1402) and advancing the barrier member distally down the ET tube (100) towards the distal end (1403) of the first shaft (404) and past the vocal cords.

In some variations, the pushing assembly may optionally further comprise a second shaft. For example, in the variation of the pushing assembly (400) shown in FIGS. 4A-4D, the pushing assembly (400) may comprise a second shaft (416). The second shaft (416) may be connected to the first shaft (404), and may be configured to assist in advancing and positioning the first shaft (404) relative to a patient's airway during use of the pushing assembly (400). For example, in some variations, the second shaft (416) may be slidably connected to the first shaft (404). The slidable connection between the second shaft (416) and the first shaft (404) may be achieved in any suitable member. In some variations, such as shown in FIGS. 4A-4D, the second shaft (416) may partially surround the first shaft (404). In some variations the first shaft (404) may comprise tracks (418) on either side of the first shaft (404), and second shaft (416) may be configured to extend into these tracks (418) to couple the second shaft (416) to the first shaft (404). The second shaft (416) may slide within the tracks (418) to allow relative movement between the first (404) and second (416) shafts. For example, the first shaft (404) may be slidable between a first position (as shown in FIGS. 4A and 4B), where the distal portion (406) of the first shaft (404) is positioned adjacent to a distal portion (420) of the second shaft (416), and a second position (as shown in FIG. 4D), where the distal portion (406) of the first shaft (404) is advanced away from the distal portion (420) of the second shaft (416).

In some variations, the second shaft may comprise a locking mechanism to temporarily prevent or resist relative movement between the first shaft (404) and the second shaft (416). In these variations, the locking mechanism may lock the first shaft (404) to the second shaft (416), such that advancement or retraction of the first shaft (404) also advances or retracts, respectively the second shaft (416), or vice versa. The locking mechanism may then be temporarily or permanently released to allow the first shaft (404) to slide relative to the second shaft (416), and vice versa. For example, FIG. 4C shows a variation of the second shaft (416) in which the second shaft comprises a locking arch (422). While shown in FIG. 4C as being positioned at a proximal end of the second shaft (416), the locking arch (422) may be positioned along any suitable portion of the length of the second shaft (416). Generally, the locking arch (422) may be configured to bias the second shaft (416) to press against first shaft (404) (for example, to press against the tracks (418) of the first shaft (404)), which may create a friction fit between the first shaft (404) and the second shaft (416). To release this connection, the locking arch (422) may be flexed away from the first shaft (404) to overcome the bias. For example, the locking arch (422) may comprise an opening (424), whereby an object may be inserted into the opening (424) to contact the interior of the locking arch (422) and flex the locking arch (422) away from the first shaft (404). In other variations, the object may break the locking arch (422) which may prevent the locking arch (422) from biasing the second shaft (416) against the first shaft (404). In some variations where a delivery device includes a holding member, the holding member may comprise a project configured to temporarily or permanently disengage the locking force provided by the locking arch (422), as will be described in more detail below.

Additionally or alternatively, the second shaft (416) may comprise an engagement cuff (426). The engagement cuff (426) may be configured to at least partially encircle an ET tube or similar device to temporarily connect the engagement cuff (426) to the ET tube or similar device. When connected to the ET tube, the engagement cuff (426) may be configured to guide the second shaft (416) along the ET tube. In some variations, the engagement cuff (426) may be configured to completely encircle the ET tube. In some of these variations, the engagement cuff (426) may be slid proximally relative to the ET tube to decouple the second shaft (416) from the ET tube. In other variations, the engagement cuff (426) may be configured to be frangible such that the engagement cuff (426) may be broken or otherwise converted into a partially-tubular member, which may allow the removal of the engagement cuff (426) anywhere along the length of the ET tube. While shown in FIGS. 4A and 4B as being positioned at a distal portion (420) of the second shaft (416), it should be appreciated that the engagement cuff (426) be positioned anywhere along the length of the second shaft (416).

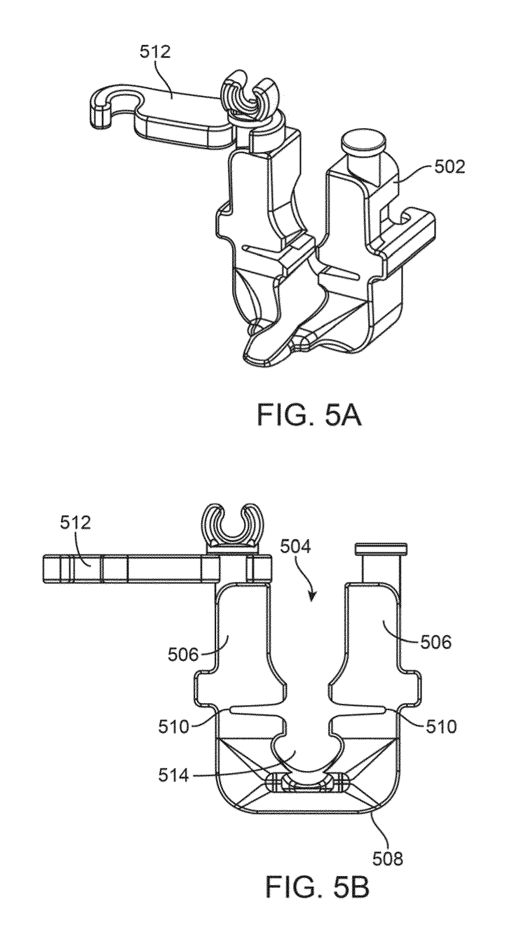

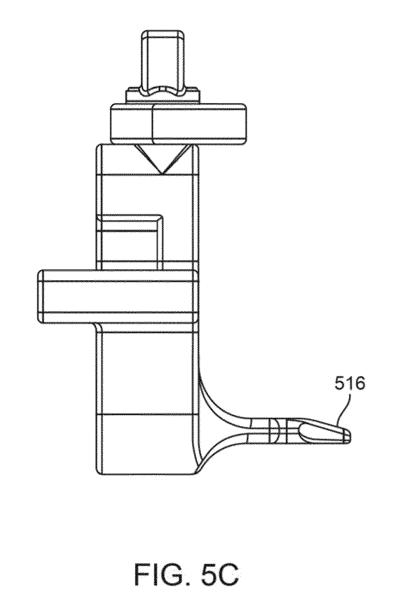

In some variations, the delivery systems described here may additionally comprise a holding member, but need not. In variations where the delivery device includes a holding member, the holding member may be configured to temporarily connect a pushing assembly of the delivery device to an ET tube or similar device. For example, FIGS. 5A-5C depict perspective, rear, and side views, respectively, of a variation of a holding member (500) suitable for use with the delivery devices described here. As shown there, the holding member (500) may comprise a body (502) which may be configured to releasably connect to an ET tube or similar device. The body (502) may be formed from one or more materials (such as, for example, silicone, polyurethane, polyethylene, polypropylene, FEP, PTFE or other plastics commonly used for machined or injection molded components).

To releasably connect the body (502) to an ET tube, the body (502) may comprise a first aperture (504) extending through the body (502). The first aperture (504) may be sized and shaped such that an ET tube may be positioned to extend through the first aperture (504). In some variations, the size of the first aperture (504) may be variable to allow the body (502) to temporarily lock the ET tube. For example, in the variation shown in FIGS. 5A-5C, the body (502) may comprise two gripping portions (506) each connected to a base member (508) by a respective hinge joint (510). While each of the two gripping portions (506) are shown in FIGS. 5A-5C as being connected to the base member (508) by hinge joints (510), in other variations only one of the two gripping portions (506) may be connected to the base member (508) by a hinge joint (510). Each hinge joint (510) may allow a respective gripping portion (506) to be flexed toward or away from another gripping portion (506) to decrease or increase, respectively, the size of the first aperture (504). For example, when an ET tube (not shown) is positioned through the first aperture (504), one or both of the gripping portions (506) may be flexed towards each other to reduce the size of the first aperture (504) and to press the gripping portions (506) against the ET tube, which in turn may cause the body (502) to be temporarily locked to the ET tube. In some variations, the body (502) may further comprise a latch (512) or other mechanism to connect and hold the two gripping portions (506) relative to each other. For example, when the gripping portions (506) are moved to lock the body (502) to the ET tube, the latch (512) may connect the gripping portions (506) to maintain the connection between the body (502) and the ET tube. Conversely, the latch (512) may be moved to a second configuration to unlatch the gripping portions (506), which may allow one or both of the gripping portions (506) to move away from each other to release the locking connection between the body (502) and the ET tube.

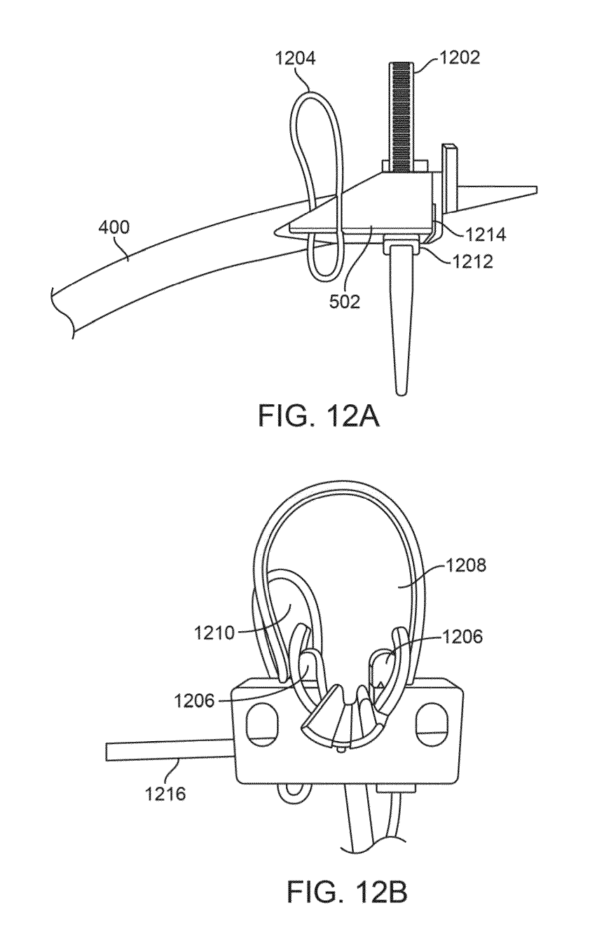

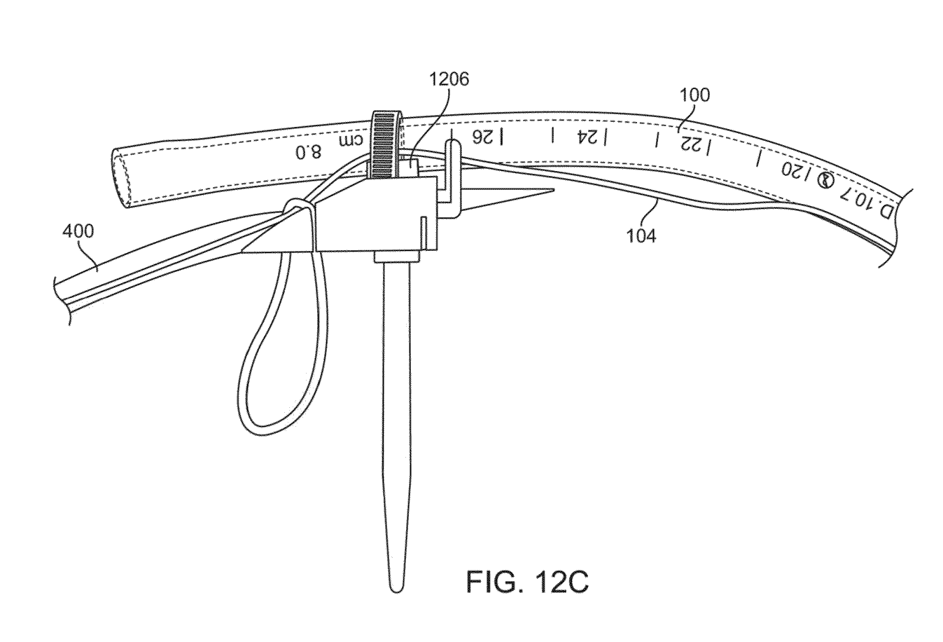

In another embodiment of the holding member (500), as shown in FIGS. 12A-12C, the holding member (500) may comprise a distal securement strap (1202) and a proximal securement strap (1204) are used to hold the endotracheal tube (100) and ET inflation tube (104) respectively to the holding member (500). The distal securement strap (1202) may form a first loop (1208) and the proximal securement strap (1204) may form a second loop (1210). During insertion, the endotracheal tube (100) and the ET inflation lumen (104) are inserted through the first loop (1208) in the securement strap (1202). The ET inflation lumen (104) is then inserted through the second loop (1210) of the proximal securement strap (1204). Prior to advancing a pushing assembly (400) along the ET (100), the straps (1202, 1204) may be tightened or adjusted to securely capture the ET tube and ET inflation tube between the respective strap and the body (502) of the holding member (500). In some variations, the holding member (500) may comprise one or more support ridges (1206) which may keep the endotracheal tube elevated above the pushing assembly (400) that runs through the body (502) of the holding member (500). This may reduce the risk of compressing the ET tube (100) onto the pushing assembly (400). The straps may be removed using a releasable lock (1212) or may be held in place permanently until cut with a blade, scissors, or the like. The holding member may also have a slot (1214) to receive a locking key (1216) to constrain the movement of the pushing assembly (400) until the locking key (1216) is removed. In some variations the first shaft may have a groove or channel, and the system may be configured such that placement of the locking key (1216) in the slot (1214) may position the locking key (1216) in the groove or channel, which may prevent movement of the first shaft relative to the locking key (1216) (and thus the holding member (500). In other instances the locking key may create a frictional engagement between the locking key (1216) and the first shaft, which may resist movement between the pushing assembly and the holding member. While discussed above as engaging a first shaft, it should be appreciated that the locking key may be configured to engage the second shaft (e.g. via frictional engagement with the second shaft and/or via a groove or channel in the second shaft).

In some variations, the body (502) may additionally comprise a second aperture (514), which may be configured to slidable receive a portion of a pushing assembly, such as those described above. When an ET tube is positioned through the first aperture (504) and a pushing assembly is positioned through second aperture (514), the holding member (500) may temporarily couple the pushing assembly relative to the ET tube. Additionally, when the holding member (500) is temporarily locked to the ET tube (such as discussed immediately above), one or more portions of the pushing assembly may be advanced or withdrawn relative to the holding member (500) to advance or withdraw, respectively, the portion or portions of the pushing assembly relative to the ET tube. In variations where the body (502) comprises first (504) and second (514) apertures, the first and second apertures may be connected (such as shown in FIGS. 5A-5C), or may be separate.

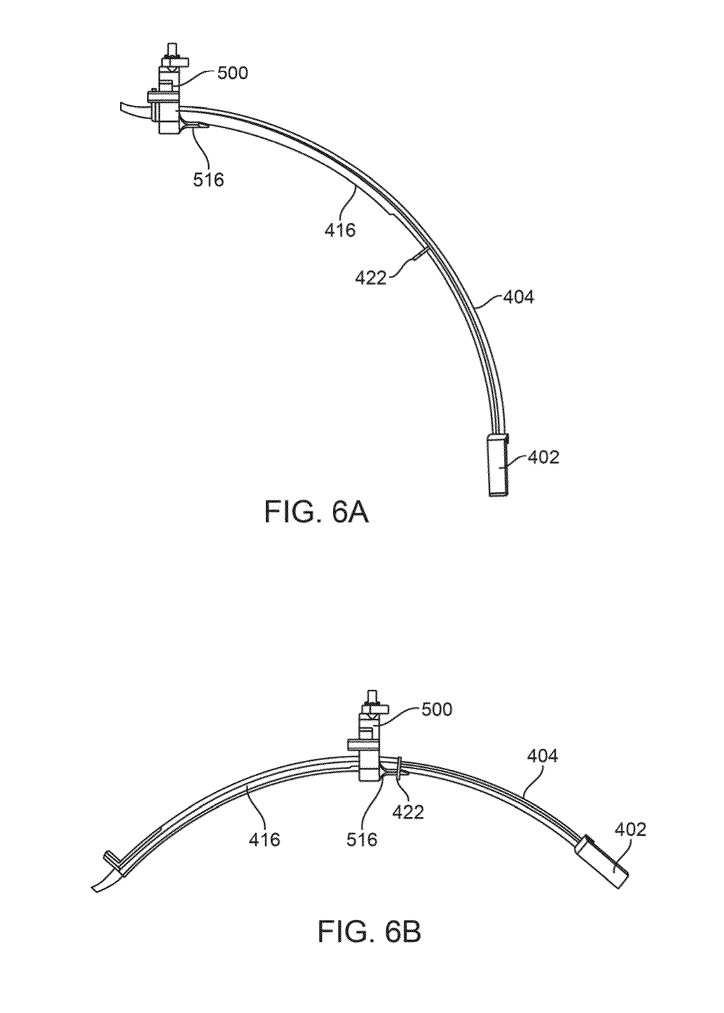

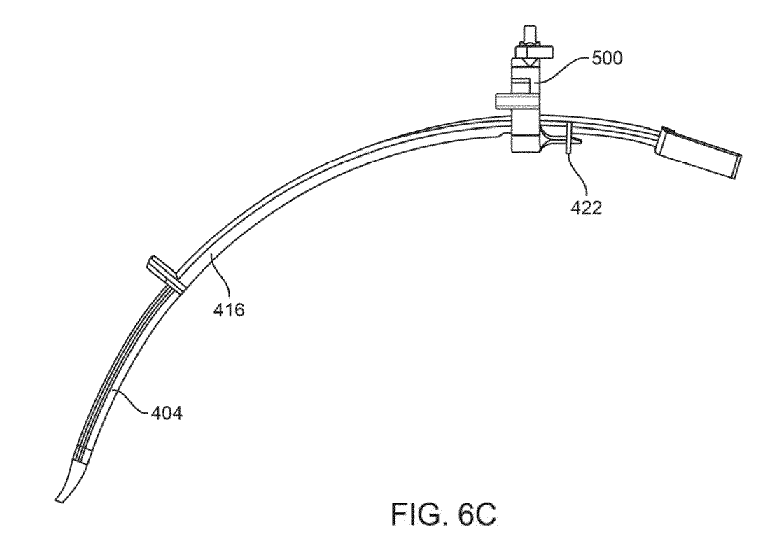

In variations where a holding member is used with a pushing assembly having a locking arch, such as the locking arch (422) described above with respect to FIGS. 4A-4D, the holding member may comprise one or more projections configured to temporarily or permanently disengage the locking arch. For example, in the variation of the holding member (500) shown in FIGS. 5A-5C, the body (502) may comprise a projection (516) extending therefrom. FIGS. 6A-6C show one manner by which the pushing assembly (400) described above with respect to FIGS. 4A-4D may be used by the holding member (500) and the projection (516) to control relative movement between the first shaft (404) and the second shaft (416). Specifically, the first shaft (404) and second shaft (416) may be slidably coupled together with the locking arch (422) fixing the first shaft (404) relative to the second shaft (416), as described in more detail above. With the first shaft (404) fixed relative the second shaft (416), the first shaft (404) and second shaft (416) may be positioned to extend through the second aperture (514) of the holding member (500), such as shown in FIG. 6A.

A pushing force may be applied to the handle (402), which may advance the first shaft (404) relative to the holding member (500). With the locking arch (422) fixing the first and second shafts, advancement of the first shaft (404) relative to the holding member (500) may also advance the second shaft (416) relative to the holding member (500). The first shaft (404) and second shaft (416) may be advanced together until the locking arch (422) reaches the projection (516), such as shown in FIG. 6B. The projection (516) may be positioned such that a portion of the projection (516) may enter the opening (424) of the locking arch (422) as the second shaft (416) is advanced. In some of these variations, at least a portion of the projection (516) may be wider than the opening (424) of the locking arch (422), such that the locking arch (422) engages the projection (516) as it is advanced. The engagement between the projection (516) and the locking arch (422) may resist further advancement of the second shaft (416) relative to the holding member (500), and may also cause the locking arch (422) to flex or break away from the first shaft (404) to temporarily or permanently release the locking force between the first shaft (404) and the second shaft (416). With the locking arch (422) disengaged, the first shaft (404) may be advanced relative to both the holding member (500) and the second shaft (416), as shown in FIG. 6C.

This two-stage advancement (advancement of the first shaft with the second shaft and advancement of the first shaft relative to the second shaft), may help position a barrier member at a target location without needing to advance all of the components of the pushing assembly into certain portions of the anatomy. For example, when a patient is intubated with an ET tube, the holding member (500) may be connected to a portion of the ET tube outside of the mouth of the patient. The engagement cuff (426) of the second shaft (416) may couple the distal portion of the second shaft (416) to the ET tube and may guide the distal portion of the second shaft (416) along the ET tube as the second shaft (416) is advanced relative to the holding member (500). This may also guide the distal portion of the first shaft (404) along the ET tube (e.g., due to the connection between the first shaft (404) and the second shaft (416)) as the first shaft (404) advances a barrier member (such as a balloon cuff). The second shaft (416) may be sized such that when the locking arch (422) reaches the projection (516) of the holding member (500), the barrier member is positioned within or past the vocal cords (e.g., due to the advancement of the first shaft (404)) but the engagement cuff (426) is positioned proximally of the vocal cords. At this point, the first shaft (404) may be advanced relative to the second shaft (416) to further advance the barrier member past the vocal cords while the engagement cuff (426) of the second shaft (416) remains outside of the trachea. Because there may be limited space between the ET tube and the vocal cords and/or trachea, keeping the engagement cuff (426) from passing through the vocal cords may reduce the likelihood that the engagement cuff (426) may catch on or otherwise become stuck in the trachea.

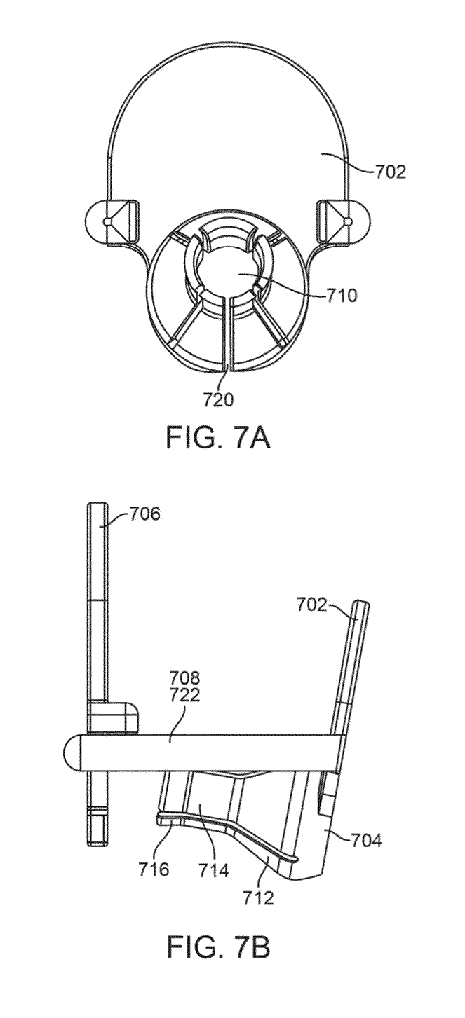

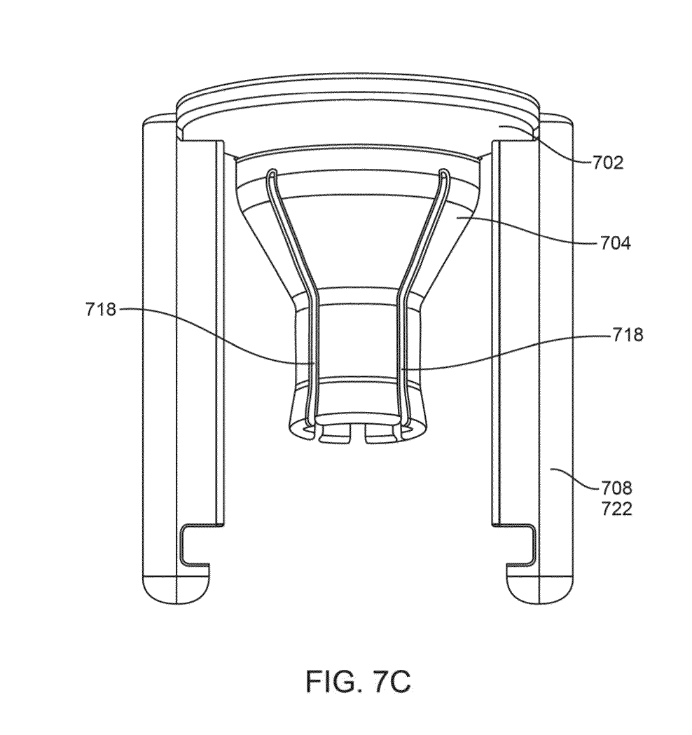

As mentioned above, the delivery devices described here may optionally include a loading assembly. When a barrier member (such as a balloon cuff) is configured to at least partially surround an ET tube or other tubular device, the loading assembly may be configured to load the barrier member around the ET tube. FIGS. 7A-7C show front, side, and top views, respectively, of a loading assembly (700) suitable for use with the delivery devices described here. As shown there, the loading assembly (700) may include a loading member (702) comprising a funnel member (704). In some variations, the loading member (702) may optional comprise a removal tab (706) and/or a connection mechanism (708) for releasably connecting the loading member (702) to a holding member, such as the holding member (500) described above with respect to FIGS. 5A-5C above.

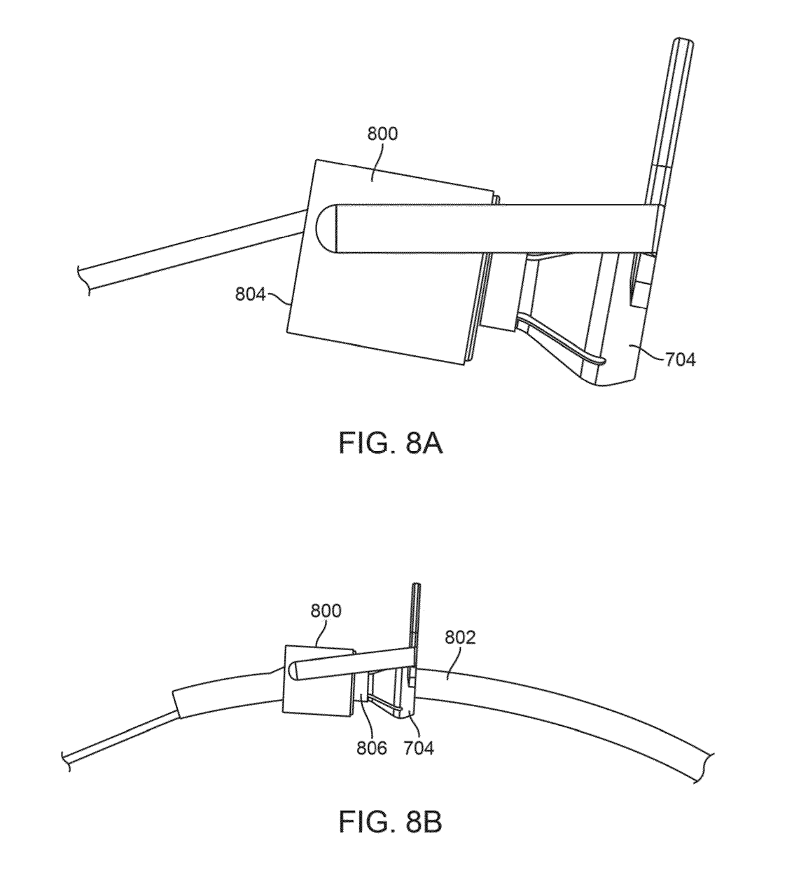

Generally, the funnel member (704) may have a lumen (710) extending through the funnel member (704), and the funnel member (704) may be used to position a barrier member around an ET tube or the like. For example, FIGS. 8A and 8B depict a manner in which the loading member (702) of FIGS. 7A-7C may be used to load a balloon cuff (800) (such as the balloon cuff (302) of the balloon cuff assembly (300) described above with respect to FIGS. 3A-3C) on an ET tube (802). As shown in FIG. 8A, the balloon cuff (800) may be positioned such that at least a portion of the balloon cuff (800) surrounds an outer portion of the funnel member (704). This may act to align a lumen (804) of the balloon cuff (802) with the lumen (710) of the funnel member (704), and in some instances may temporarily couple the balloon cuff (800) to the funnel member (704). Any suitable portion or portions of the balloon cuff (800) may be positioned around the funnel member (704). For example, in variations where the balloon cuff (800) comprises a sealing sleeve (806), such as described above, the sealing sleeve (806) may be positioned around the funnel member (704). In some of these variations, the sealing sleeve (806) may conform to outer periphery of the funnel member (704), which may help temporarily connect the balloon cuff (802) and the funnel member (704).

With the balloon cuff (800) positioned around a first end the funnel member (704), the ET tube (802) may be advanced through an opposite end of the funnel member (704) and into the lumen (710) of the funnel member (704). The ET tube (802) may be further advanced such that the ET tube extends through the lumen (710) of the funnel member (704) and out of the first end of the funnel member (704). As the ET tube (802) is advanced out of the first end of the funnel member (704), it may enter the lumen (804) of the balloon cuff (800), thereby positioning the balloon cuff (800) around the ET tube (802), as shown in FIG. 8B. With the balloon cuff (800) loaded on the ET tube (802), the loading member (702) may be removed from the ET tube (802), such as discussed in more detail below.

Generally, the funnel member (704) may have any suitable profile. For example, in the variation of the funnel member (704) shown in FIGS. 7A-7C, the funnel member (704) may comprise a tapered portion (712) and a stem portion (714) extending from the tapered portion (712). In these variations, the diameter of the lumen (710) of the funnel member (704) may be substantially constant in the stem portion (714), and may increase across the tapered portion (712) between the stem portion (714) and the second end of the funnel member (704). When an ET tube or other device is advanced into the second end of the funnel member (704), the tapering diameter of the lumen (710) may help guide the ET tube towards the stem portion (714) of the funnel member (704).

In some variations, the outer diameter of the stem portion (714) may be substantially constant, and the outer diameter of the tapered portion (712) may increase along the length of the tapered portion (712). In some of these variations, when a barrier member such as a balloon cuff is positioned around an exterior portion of the funnel member (704), the barrier member may be positioned around the stem portion (714). In some of these variations, the increasing diameter of the tapered portion (712) may limit advancement of the barrier member relative to the funnel member (704).

In some variations, the funnel member (704) may further comprise a flared portion (716), but need not. For example, in the variation of the funnel member (704) shown in FIGS. 7A-7C, the funnel member (704) may have a tapered portion (712), a flared portion (716), and a stem portion (714) positioned between the tapered portion (712) and the flared portion (716). As shown there, the outer diameter of stem portion (714) may have an outer diameter that is less than the diameters of the tapered portion (712) and the flared portion (716). Specifically, the outer diameter of the tapered portion (712) may increase away from the stem portion (714) (e.g., toward the second end of the funnel member (714)). The outer diameter of the flared portion (716) may also increase away from the stem portion (714) (e.g., toward the first end of the funnel member (714)). When a portion of a barrier member is positioned around the stem portion (714), the flared portion (716) may resist withdrawal of the barrier member relative to the funnel member (714).

For example, when the balloon cuff (800) is positioned around the funnel portion (704) as discussed above with respect to FIGS. 8A and 8B, the sealing sleeve (806) may be positioned around the stem portion (714) of the funnel member (704). In some of these variations, the sealing sleeve (806) may be sized to have a diameter less than or equal to the outer diameter of the stem portion (714), such that the sealing sleeve (806) may conform closely to the stem portion (714). In these variations, the sealing sleeve (806) may resist withdrawal of the balloon cuff (800) away from the funnel member (704), as the sealing sleeve (806) may resist being pulled over the larger outer diameter of the flared portion (716). Similarly, the sealing sleeve (806) may resist being advanced along the larger diameter of the tapered portion (712).

It should be appreciated that the profile of the funnel member (704) may include any combination of tapered portions, stem portions, and flared portions. For example, in some variations, the funnel member (704) may comprise only a tapered portion. In other variations, the funnel member (704) may include a tapered portion and a stem portion extending therefrom, but may not comprise a flared portion. In still other variations, the funnel member (704) may include a tapered portion with a flared portion extending therefrom, but does not comprise a stem portion.

In some variations, the funnel member (704) may be configured to be expandable. When an ET tube or other device is inserted through the lumen (710) of the funnel member (704), the ET tube may be larger than a portion of the lumen (710), such that advancement of the ET tube may cause the funnel member (704) to expand in order to accommodate the ET tube. The funnel member (704) may be configured to expand in any suitable manner. In some variations, one or more portions of the funnel member (704) may be formed from a flexible or deformable material, such that the funnel member (704) may stretch or otherwise deform as the ET tube is advanced therethrough. Additionally or alternatively, the funnel member (704) may comprise one or more slots. For example, in the variation of the funnel member (704) shown in FIGS. 7A-7C, the funnel member (704) may comprise one or more slots (718). In these variations, the advancement of an ET tube through the funnel member (704) may cause the segments of the funnel member (704) between the slots (718) to flex away from each other, thereby increasing the size of the slots (718) and the diameter of the lumen (710).

When an expandable funnel member (704) is used to load a balloon cuff having a sealing sleeve onto an ET tube (such as the balloon cuff (800) with sealing sleeve (806) and ET tube (802) described above with respect to FIGS. 8A and 8B)), expansion of the funnel member (704) may temporarily stretch the sealing sleeve to accommodate the ET tube in the sealing sleeve. When the funnel member (704) is removed, the sealing sleeve may contract around the ET tube to conform to the ET tube and provide a seal between the balloon cuff and the ET tube, such as described in more detail above.

As mentioned above, once the funnel member (704) is used to load a barrier member on an ET tube or similar device, the funnel member may be removed from the ET tube. In some variations, the funnel member may be configured to be removed from the ET tube at any point along the length of the ET tube. In some variations, a portion of the funnel member (704) may be frangible, such that the funnel member (704) may be broken or otherwise converted into a partially-tubular member. In other variations where the funnel member (704) comprises one or more slots (718), one or more of the slots (718) may extend along the entire length of the funnel member (704), such that the ET tube may be pulled through the slot. For example, in the variation of the funnel member (704) shown in FIGS. 107A-7C, the funnel member (704) may comprise a slot (720) extending the entire length of the funnel member (704), but need not. In these variations, a tube positioned in the lumen (710) of the funnel member (704) may be removed through the slot (720).

In some variations of the loading members described here, the loading members may be configured to connect to the delivery device. For example, in variations where the delivery device comprises a holding member, the loading member may be configured to connect to the holding member. For example, in the variation of the loading member (702) shown in FIGS. 7A-7C, the loading member (702) may comprise a connection mechanism (708). The connection mechanism (708) may be any suitable mechanism for connecting the loading member (702) to a holding member. In the variation of the loading member (702) shown in FIGS. 7A-7C, the connection mechanism (708) may comprise connection rods (722), which in turn may connect the loading member (702) to a holding member.

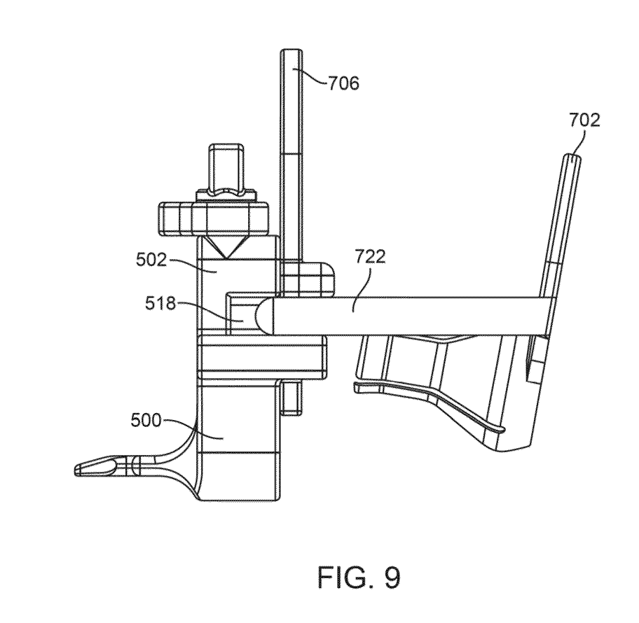

FIG. 9 shows a manner by which the connection rods (722) of the loading member (702) of FIGS. 7A-7C may connect the loading member (702) to the variation of the holding member (500) described above with respect to FIGS. 5A-5C. As shown there, the holding member (500) may comprise recesses (518) in the body (502). The connection rods (722) may be positioned at least partially in respective recesses (518) to position and hold the body (502) of the holding member (500) between the connection rods (722), which may thereby temporarily connect the loading member (702) to the holding member (500). In some variations, the loading member (702) may be pulled away from holding member (500) to pull the connection rods (722) away from the holding member (500), which may in turn disengage the loading member (702) from the holding member (500). For example, in variations where the loading member (702) comprises a removal tab (706), the removal tab (706) may provide a structure for a user to manipulate to connect and disconnect the loading member (702) from the holding member (500). In variations where the loading member (702) does not include a removal tab (706), a user may grasp any other portion of the loading member (702) to connect or disconnect the loading member (702) from the holding member (500).

While the connection mechanism (708) is shown in FIGS. 7A-7C and 9 as comprising connecting rods, the connection mechanism (708) may comprise any suitable mechanism for connecting the loading member (702) to a holding member. In some variations, the holding member may comprise one or more rods which may be received by one or more recesses of a loading member.

As mentioned above, the methods described here may comprise advancing a barrier member within or along a patient's airway to form a barrier therein. In some variations, the barrier member may comprise a balloon cuff assembly, which may comprise a balloon cuff such as described above. For example, in some variations the balloon cuff may comprise a support ring and a balloon. The balloon cuff assembly may further comprise an inflation tube and an inflation port, such that the inflatable member may be inflated by introducing a gas or fluid into the inflation port (or may be deflated by withdrawing fluid therefrom).

In some variations, the barrier member may be positioned around an ET tube or laryngeal mask at a location along the patient's airway. In some instances, the barrier member may be positioned simultaneously with the ET tube or laryngeal mask. In other instances, the barrier member may be advanced along a pre-placed ET tube or laryngeal mask. The barrier member may be advanced using one or more of the delivery devices described above. In these variations, the barrier member may be temporarily attached to the delivery device. In variations where the delivery device comprises a loading assembly, such as described above, a portion of the barrier member may be temporarily attached to the loading assembly. Additionally or alternatively, the barrier member may be temporarily connected to a pushing assembly of the delivery device.

The barrier member may be loaded onto the ET tube or laryngeal mask, and the delivery device may be manipulated to advance the barrier member. For example, in variations where the delivery device comprises a loading assembly, the loading assembly may be used to help load the barrier member onto the ET tube or laryngeal mask. Advancing the barrier member may comprise connecting a holding member (in variations where the delivery device comprises a holding member) to the ET tube or laryngeal mask, and advancing the pushing assembly relative to the holding member to advance the barrier member along the ET tube or laryngeal mask. Advancement of the deployable member may be done without direct visualization of the vocal cords, and the delivery instrument may provide tactile feedback and/or other feedback to indicate placement of the deployable member.

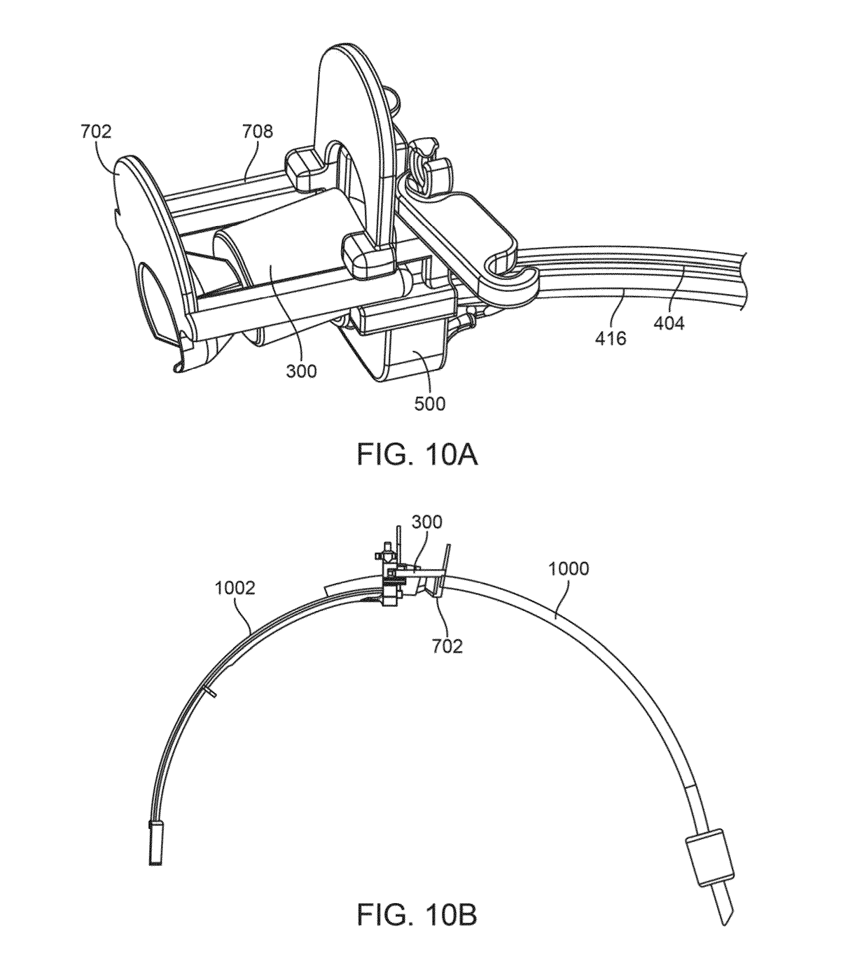

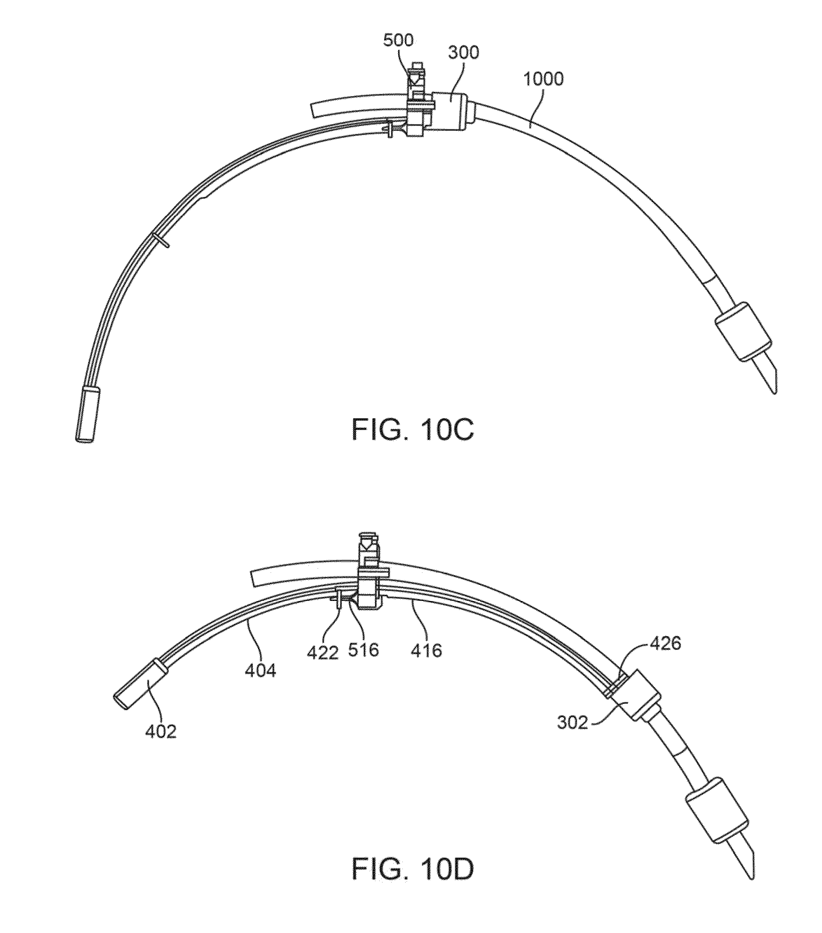

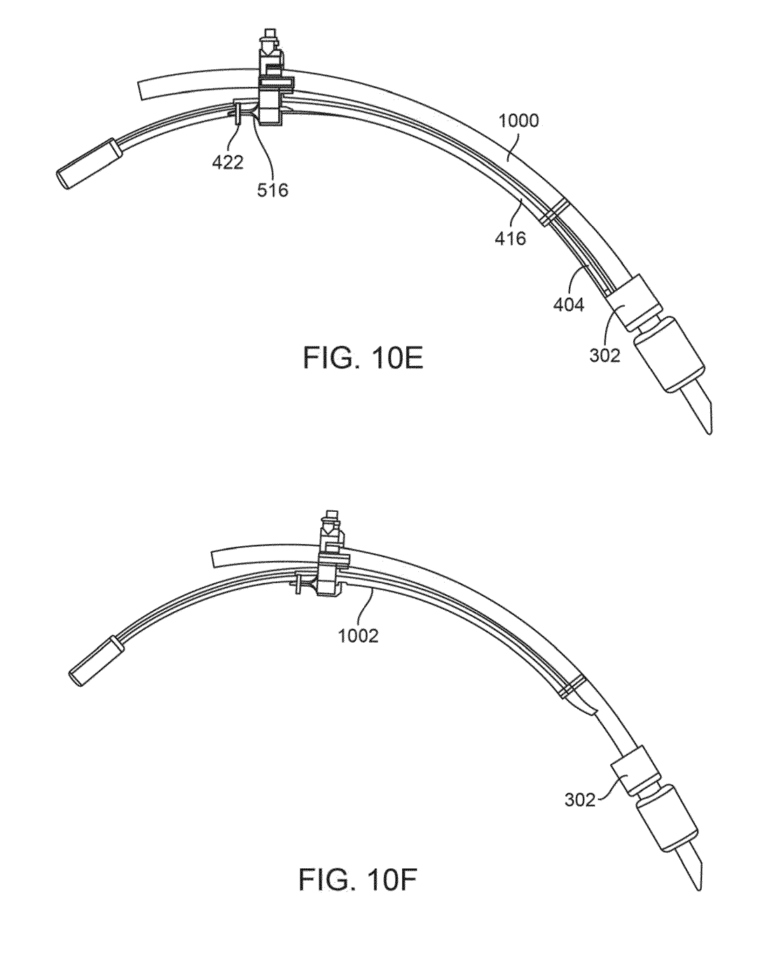

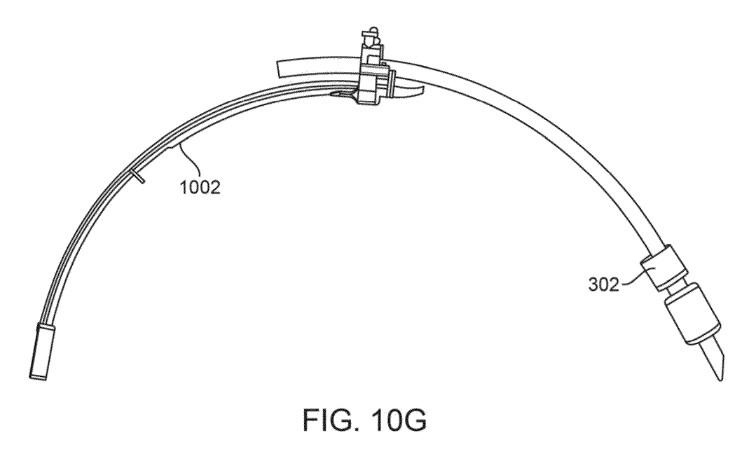

FIGS. 10A-10G illustrate an example of how the balloon cuff assembly (300) described above with respect to FIGS. 3A-3B may be advanced along an ET tube (1000) positioned in a patient using a delivery device (1002). The ET tube (1000) may be positioned such that an ET balloon (1004) of the ET balloon is position within the trachea past the vocal cords. For the purposes of illustration, FIGS. 10A-10G depict the method steps but do not depict the patient. The delivery device (1002) may include the pushing assembly (400) described above with respect to FIGS. 4A-4D, the holding member (500) described above with respect to FIGS. 5A-5C, and the loading assembly (700) described above with respect to FIGS. 7A-7C (which are labeled in FIGS. 10A-10G as in these figures).

The delivery device (1002) assembled, and the balloon cuff assembly (300) may be connected to the delivery device (1002), as shown in FIG. 10A. The loading member (702) may be connected to the holding member (500) (e.g., via the connection mechanism (708)), and the holding member (500) may be slidably connected to the pushing assembly (400), such as described above (e.g., the first shaft (404) and the second shaft (416) may be positioned in the second aperture (514) of the body (502)). The holding member (500) and loading member (702) may be positioned at a distal portion of the pushing assembly (400).

The balloon cuff assembly (300) may, in turn, be connected to the pushing assembly (400) and the loading assembly (700). For example, a portion of balloon cuff (302) (e.g., the sealing sleeve (312)) may be positioned around a portion of funnel member (704) (e.g., the stem portion (714) of the funnel member (704)). This may align the lumen (314) of the balloon cuff (302) with the lumen (710) of the funnel member (702), and the connection between the loading assembly (700) and the holding member (500) may also align the lumen (314) of the balloon cuff (302) with the first aperture (504) of the body (502). The pushing assembly (400) may be positioned relative to the balloon cuff assembly (300), such that the distal portion (406) of the first shaft (404) engages the balloon cuff (302) (e.g., the support ring (310) of the balloon cuff (302)) and the inflation port (304) is connected to the handle (402) (e.g., via insertion of the inflation port (304) into the recess (410) of the handle (402), which may position the inflation tube (306) along the first shaft (404) (e.g., along the channel (408) in the first shaft (404)).

With the balloon cuff assembly (300) connected to the delivery device (1002), the delivery device (1002) may be connected to the ET tube (1000). A proximal end of the ET tube (1000) may be inserted into the lumen (710) of the funnel member (702), the lumen (314) of the balloon cuff (302), and the first aperture (504) of the body (502), as shown in FIG. 10B. This may also comprise connecting the engagement cuff (426) of the second shaft (416) to the ET tube (1000). The funnel member (702) may be disconnected from the ET tube (1000) and the delivery device (1002), which may thereby leave the balloon cuff (302) and the holding member (500) connected to a proximal portion of the ET tube (1000), as shown in FIG. 10C. The holding member (500) may be locked to the ET tube (1000), such as discussed above, and the first (404) and second (416) shafts may be advanced relative to the holding member (500) to advance the balloon cuff (302) relative to the ET tube (1000).

The first (404) and second (416) shafts may be advanced together (e.g., by applying a pushing force to the handle (402)) until the locking arch (422) reaches the projection (516) of the holding member (500), as shown in FIG. 10D. In some variations, this may position the engagement cuff (426) of the second shaft (416) in the oropharyngeal space proximal of the vocal cords (VC), and may position the balloon cuff (302) at least partially past the vocal cords (VC). Further advancement of the first shaft (404) may cause the projection (516) to engage the locking arch (422) to release the locking force provided by the locking arch (422), as discussed above, and to prevent further advancement of the second shaft (416) relative to the ET tube. The first shaft (404) may be advanced relative to the second shaft (416), the holding member (500), and the ET tube (1000) to further advance the balloon cuff (302) into the trachea (TR), as shown in FIG. 10E. The delivery device (1002) may be removed from the patient (FIGS. 10F AND 10G), and the balloon cuff (302) may be inflated to form a barrier in the trachea (TR).

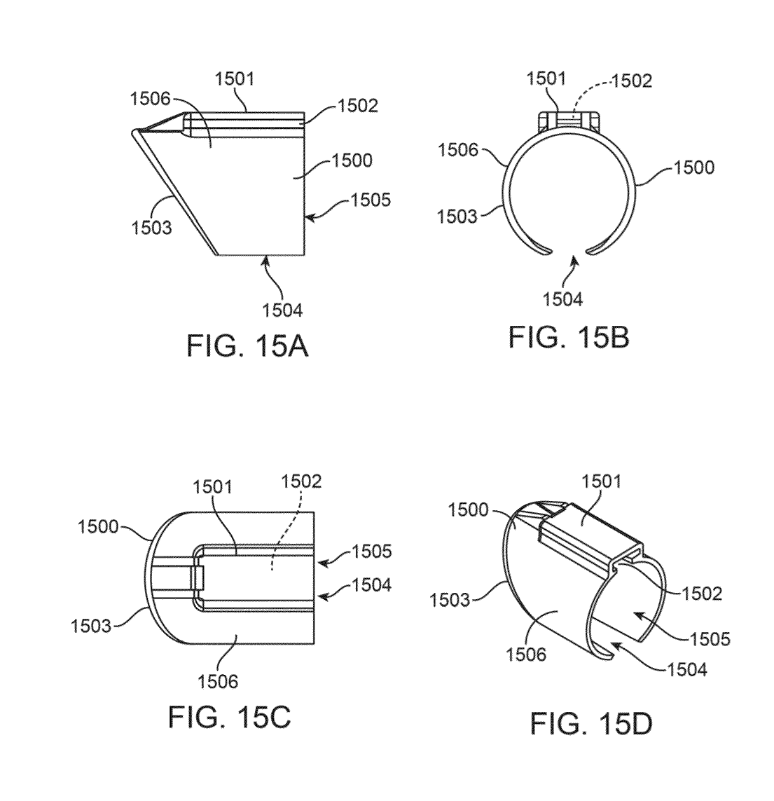

In yet another variation of the balloon cuff assembly, FIGS. 15A to 15D depict respective side, end, top, perspective views of an assembly which is configured to facilitate movement along the endotracheal tube as well as access into the patient's body. This variation of the balloon cuff assembly (1500) has a support ring body (1506) which may be formed into a partial tubular shape defining a tube engagement channel (1505) which may receive the endotracheal tube, as previously described. While the channel (1505) may be formed from a support ring body (1506) which is continuous, an opening (1504) may be optionally formed along the support ring body (1506) through which the assembly (1500) may be mounted or secured upon the tube for delivery into the patient body. Opposite to the opening (1504), a pusher engagement (1501) feature may be located which extends longitudinally along the support ring body (1506) and defines a pusher receiving channel (1502) that is configured to receive a shaft from the pushing assembly.

A distal edge (1503) of the support ring body (1506) may be optionally tapered to angle from the distal end of the pusher engagement (1501) towards the opening (1504) to facilitate advancement of the assembly (1500) into the patient' body. However, the tapered distal edge (1503) may instead be perpendicularly defined relative to the support ring body (1506) or the taper may be defined at various angles depending upon the desired degree of taper.

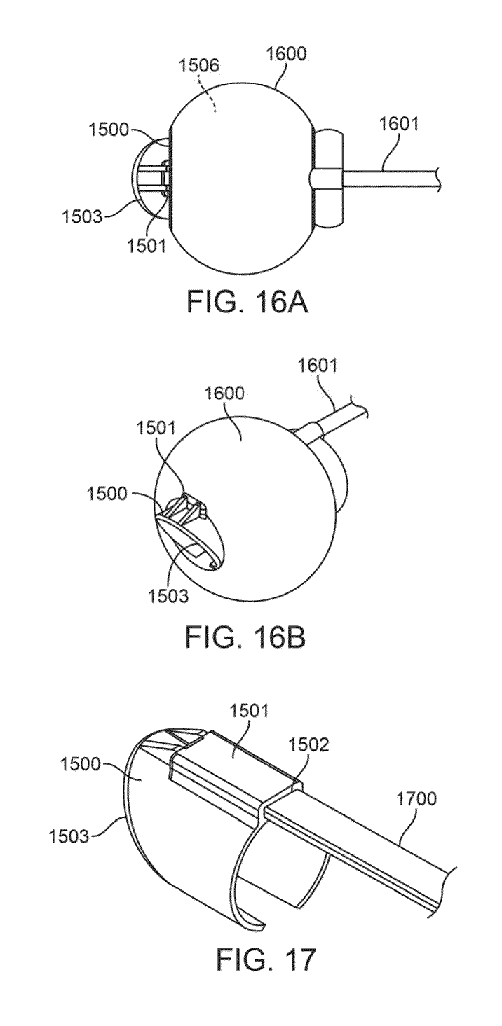

In either case, the balloon (1600) may be formed integrally with the support ring body (1506) or the balloon (1600) may be formed separately and connected, e.g. via bonding, welding, or the like. The balloon (1600) may be formed to extend entirely around the circumference of the support ring body (1506) including over the opening (1504), as shown in the top and perspective views of the assembly (1500) of FIGS. 16A and 16B, while in other embodiments the balloon (1600) may be configured to extend partially around the support ring body (1506). In order to inflate balloon (1600), inflation tube (1601) may be fluidly connected to the balloon (1600) to desirably inflate and deflate as needed.

As described herein, the assembly (1500) may be advanced distally over an endotracheal tube by urging the assembly (1500), e.g., via a second shaft (1700). In utilizing the variation of assembly (1500), a second shaft (1700) may be configured as a relatively flattened shaft such as a ribbon member, as shown in the perspective view of FIG. 17. Second shaft (1700) may be configured as a ribbon member where its width is relatively longer than its height such that the second shaft (1700) presents a low-profile when advanced along an endotracheal tube. Having a relatively low-profile may prevent unnecessary trauma to the patient tissue during deployment but since second shaft (1700) may be fabricated from a relatively rigid material (e.g., stainless steel, plastics, etc.), the second shaft (1700) may still provide structural support for advancing (or retracting) the assembly (1500) into or from the patient via the second shaft (1700).

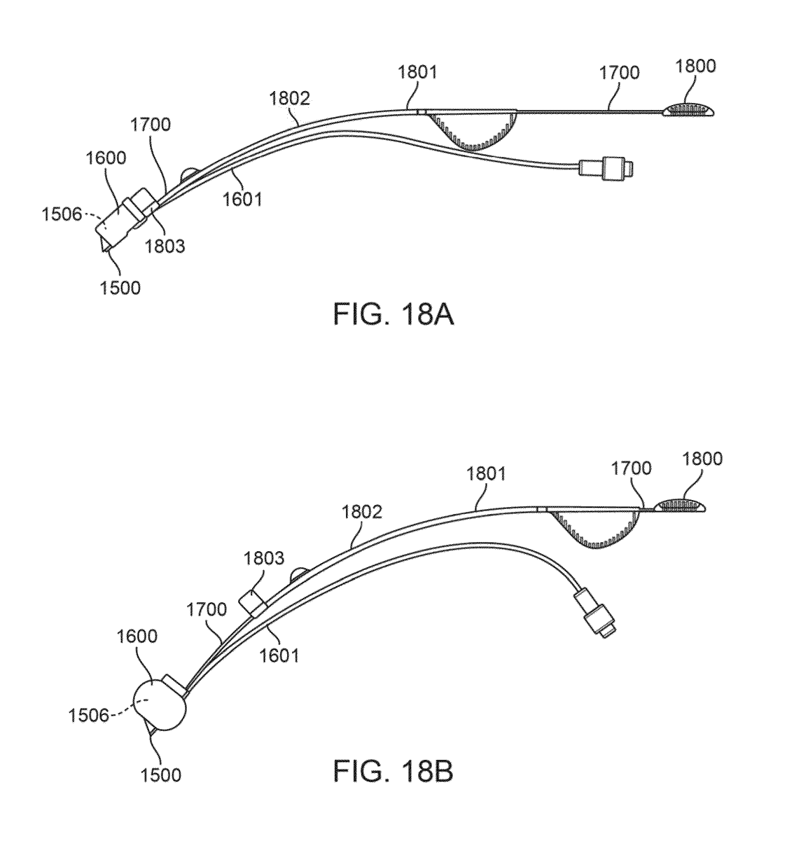

In use, the assembly (1500) may be engaged with a pusher assembly for deploying into the patient. FIGS. 18A and 18B depict side views of an example of a pushing assembly having a balloon cuff assembly engaged and advanced distally by the first shaft (1801) and second shaft (1700). With the assembly (1500) engaged with the second shaft (1700), as described above, the balloon (1600) may be in its deflated state and a proximal end of the support ring body (1506) may be abutted or positioned adjacent to a cuff engagement member (1803) located proximal to the support ring body (1506), as shown in the side view of FIG. 18A. The second shaft (1700) may be slidably positioned within an engagement channel (1802) defined along the first shaft (1801) such that prior to deployment of the assembly (1500), the second shaft (1700) may remain retracted relative to the first shaft (1801) and the assembly (1500) may be abutted adjacent to the cuff engagement member (1803).

Once the assembly (1500) has been slidingly engaged or secured to the endotracheal tube, first shaft (1801) may be advanced distally along the tube to advance assembly (1500) and subsequently, second shaft (1700) may then be advanced relative to the first shaft (1801) by urging the second shaft (1700) to slide within the engagement channel (1802) via second shaft handle (1800) to further advance the assembly (1500), as described in further detail above. Once the assembly (1500) has been suitably positioned, balloon (1600) may be inflated or expanded, as shown in FIG. 18B, and the cuff engagement member (1803) may be disengaged from the support ring body (1506) and removed from the body.

The applications of the devices and methods discussed above are not limited to treatment of conditions associated with ET tubes and esophageal masks. The systems and methods described above may be used to place a barrier member along a nasogastric or orogastric tubes to create a seal in the oropharyngeal space, esophagus, or stomach. Moreover, such systems and methods may be applied to other treatment sites within the body, e.g., esophagus, urinary tract, etc. Modification of the above-described assemblies and methods for carrying out the invention, combinations between different variations as practicable, and variations of aspects of the invention that are obvious to those of skill in the art are intended to be within the scope of the claims.

What is claimed is:

1. A barrier apparatus for use with an endotracheal tube, comprising:
an expandable barrier assembly configured to be positioned over or around an endotracheal tube;
a pushing assembly having a handle and a first shaft extending therefrom, the first shaft having a length and a distal end configured to engage at least a portion of the barrier assembly; and
a holding member securable to the endotracheal tube and slidingly engagable with the pushing assembly.

2. The apparatus of claim 1 further comprising a loading assembly connectable to the holding member and configured to load the barrier assembly around the endotracheal tube.

3. The apparatus of claim 2 wherein the loading assembly comprises a funnel member.

4. The apparatus of claim 2 wherein the loading assembly is releasably connectable to the barrier assembly.

5. The apparatus of claim 1 wherein the barrier assembly comprises a barrier which is expandable into an obstructing configuration sized to conform to an airway of a patient.

6. The apparatus of claim 1 wherein the barrier assembly comprises a balloon cuff assembly having an inflatable balloon mounted on a support member which defines a lumen extending therethrough.

7. The apparatus of claim 6 wherein the support member comprises a support ring attached to the inflatable balloon.

8. The apparatus of claim 6 wherein the barrier assembly further comprises a sealing sleeve attached to the support member and/or inflatable balloon.

9. The apparatus of claim 1 wherein the first shaft defines a slot along at least a portion of the length of the first shaft, the slot being sized for receiving an inflation port in communication with the barrier assembly.

10. The apparatus of claim 1 wherein the distal end of the first shaft comprises a forked member defining at least one slot such that the forked member is configured to contact and press against one or more portions of the barrier assembly.

11. The apparatus of claim 10 wherein the forked member defines a radius of curvature approximating a curvature of the barrier assembly.

12. The apparatus of claim 1 wherein the distal end of the first shaft comprises an adjustable loop extending from the distal end.

13. The apparatus of claim 1 wherein the distal end of the first shaft comprises a partial or complete ring member.

14. The apparatus of claim 1 further comprising a second shaft slidably connected to the first shaft.

15. The apparatus of claim 14 wherein the second shaft is slidably extendable from the first shaft from a first retracted position where the distal end of the first shaft and a distal end of the second shaft are adjacent to a second extended position where the distal end of the first shaft is advanced away from the distal end of the second shaft.

16. The apparatus of claim 14 further comprising a releasable locking mechanism configured to lock the first and second shafts to one another.

17. The apparatus of claim 14 further comprising an engagement cuff extending from the second shaft, the engagement cuff having a configuration to slidingly engage a portion of the endotracheal tube.

18. The apparatus of claim 14 wherein the second shaft is comprised of a ribbon member having a low-profile.

19. The apparatus of claim 1 further comprising a holding member configured to connect the pushing assembly to a portion of the endotracheal tube.

20. The apparatus of claim 19 wherein the holding member further comprises a projection extending therefrom which is configured to control relative movement between the first shaft and a second shaft.

21. The apparatus of claim 1 further comprising one or more cables slidably extending along the first shaft and coupled to the barrier assembly wherein tension on the one or more cables actuates the barrier assembly along the endotracheal tube.

22. The apparatus of claim 1 wherein the length of the first shaft defines a curvature configured to conform to an airway of a patient.

23. A method for deploying a barrier apparatus, comprising:
securing a holding member along a portion of an endotracheal tube extending from a mouth of a patient;
urging a first shaft distally relative to the holding member such that the first shaft follows the endotracheal tube until a barrier assembly is positioned via the first shaft in proximity to or at least partially past vocal cords of the patient; and,
further advancing the first shaft relative to the holding member such that the barrier assembly is positioned distal to the vocal cords of the patient.

24. The method of claim 23 further comprising expanding the barrier assembly such that the barrier assembly expands between an outer surface of the endotracheal tube and an airway of the patient.

25. The method of claim 23 further comprising removing the first shaft from the barrier assembly.

26. The method of claim 23 further comprising removing the barrier assembly and the endotracheal tube from the patient simultaneously.

27. The method of claim 23 wherein securing a holding member comprises securing the holding member at a predetermined position along the endotracheal tube.

28. The method of claim 23 wherein urging a first shaft comprises urging the first shaft and a second shaft distally relative to the holding member such that the first and second shafts follow the endotracheal tube.

29. The method of claim 28 wherein urging the first shaft and the second shaft comprises advancing each shaft simultaneously.

30. The method of claim 28 wherein urging the first shaft and the second shaft comprises sliding the barrier assembly over the endotracheal tube until the barrier assembly is positioned at least partially past the vocal cords.

31. The method of claim 23 wherein urging further comprises aligning a lumen of the barrier assembly with the holding member.

32. The method of claim 28 further comprising releasing the first shaft from the second shaft prior to further advancing the first shaft.

33. The method of claim 32 wherein releasing the first shaft from the second shaft comprises engaging a locking arch along the second shaft via a projection extending from the holding member.

34. The method of claim 23 further comprising securing the barrier assembly to the endotracheal tube.

35. The method of claim 23 wherein prior to securing a holding member, advancing the endotracheal tube through the mouth and within a trachea of the patient such that an endotracheal balloon is positioned distal to the distal cords of the patient.
